(19) United States
(12) Patent Application Publication
(10) Pub. No.: US 2026/0283988 A1
(43) Pub. Date: Sep. 24, 2026

(54) FERROPTOSIS INHIBITORS

(71) Applicant: HELMHOLTZ ZENTRUM MÜNCHEN DEUTSCHES FORSCHUNGSZENTRUM FÜR GESUNDHEIT UND UMWELT (GMBH), Neuherberg (DE)

(72) Inventors: Kamyar HADIAN, Neuherberg (DE); Juliane TSCHUCK, Neuherberg (DE); Manfred RÖSNER, Eppstein/Ts (DE)

(73) Assignee: HELMHOLTZ ZENTRUM MÜNCHEN DEUTSCHES FORSCHUNGSZENTRUM FÜR GESUNDHEIT UND UMWELT (GMBH), (omitted)

(21) Appl. No.: 19/477,055

(22) PCT Filed: Apr. 19, 2024
(86) PCT No.: PCT/EP2024/060769

§ 371 Date: Oct. 20, 2025

(30) Foreign Application Priority Data
Apr. 21, 2023 (EP) 23169117.1

(51) Int. Cl. A61K 31/192; A61K 31/122; A61K 31/136; A61K 31/165; A61K 31/167; A61K 31/196; A61K 31/416; C07C 225/30; C07C 229/18; C07C 233/43; C07C 237/06

(52) U.S. Cl. CPC A61K 31/192; A61K 31/122; A61K 31/136; A61K 31/165; A61K 31/167; A61K 31/196; A61K 31/416; C07C 225/30; C07C 229/18; C07C 233/43; C07C 237/06; C07C 2602/10

(57) ABSTRACT

The invention is directed to a quinone for use in the inhibition of ferroptosis.

### TECHNICAL FIELD OF THE INVENTION

The present invention relates to a quinone for use in the inhibition of ferroptosis.

### BACKGROUND ART

Ferroptosis is a regulated and non-apoptotic form of cell death. Ferroptotic cell death is characterized by extensive iron-dependent lipid peroxidation, which can be the cause for distinct degenerative diseases (1). To prevent dying from ferroptosis, cells have developed a manifold of ferroptosis-inhibitory mechanisms: the system x_{c}⁻/glutathione/GPX4 axis (2), the FSP1-ubiquinol axis (3, 4), the GCH1/tetrahydrobiopterin/DHFR axis (5, 6), the DHODH/ubiquinol axis (7), and the FSP1/vitamin-K axis (8). All these inhibitory modules revert lipid peroxides to their non-lethal alcohol forms and thereby counteract ferroptotic cell death. Ferroptosis can be chemically triggered by inhibiting system x_{c}⁻ (e.g., erastin or IKE), or directly inhibiting GPX4 (e.g., (1S,3R)-RSL3 or ML210), or FIN56 as well as FINO₂ that both have mixed effects to cause ferroptosis (9). In contrast, numerous radical-trapping antioxidants (RTAs) such as ferrostatin-1, liproxstatins-1, and vitamin E are capable of suppressing lipid peroxidation (2, 9), thereby inhibiting ferroptosis. Despite the availability of many ferroptosis-modulating compounds, most of them are either not in vivo compatible or still in preclinical development (10); thus, raising the need for additional strategies to develop ferroptosis-based medicines (11).

Research of the past years have shed light to the occurrence of ferroptosis in organ injury and degenerative diseases of the brain, kidney, heart, and others. Hence, ferroptosis inhibition may prove therapeutically beneficial to treat the aforementioned diseases.

### SUMMARY OF THE INVENTION

The invention is directed to a quinone for use in the inhibition of ferroptosis.

Further, the invention is directed to a quinone of claim for use in the treatment of a disease which can be treated or prevented by inhibition of ferroptosis, in particular the disease is selected from the group consisting of Amyotrophic Lateral Sclerosis (ALS); Alzheimer's Disease (AD); Parkinson's Disease (PD); Huntington's Disease (HD); COPD; ischemia reperfusion injury (IRI) of liver, kidney, intestine, lung; myocardial infarction; cardiomyopathy; stroke; traumatic brain injury; kidney degeneration; retinal degeneration (2, 10, 11).

The invention is directed to quinones, wherein in one embodiment the quinone for use in said treatment is selected from a quinone as specified in one of the general formula (I) or (II)

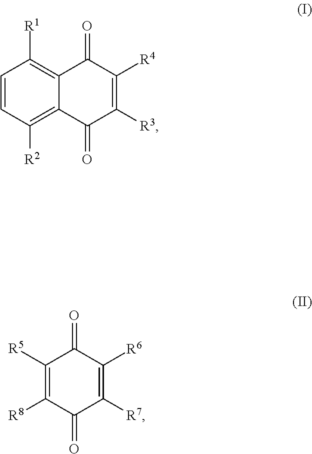

R¹ and R² are independently selected from the group consisting of H and —OH;
R³, R⁵, R⁷ and R⁸ are independently selected from the group consisting of H, —(C₁-C₃₀)alkyl, —(C₂-C₁₂)alkenyl, —OH, chlorine, —O(C₁-C₁₀)alkyl, trifluoromethyl;
R⁴ is independently selected from the group consisting of —(C₁-C₃₀)alkyl, —(C₂-C₁₂)alkenyl, —NH(C₁-C₁₀)alkyl and —NH(C₆-C₁₂)aryl,

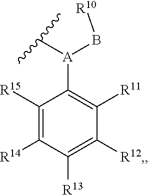

wherein in (C₁-C₃₀)alkyl, —(C₂-C₁₂)alkenyl, —NH(C₁-C₁₀)alkyl and —NH(C₆-C₁₂)aryl one or more hydrogen atoms are optionally independently substituted by a substituent selected from the group consisting of —OH, —O(C₁-C₁₀)alkyl, (C₁-C₁₀)alkyl, —C(O)(C₁-C₁₀)alkyl, —COO(C₁-C₁₀)alkyl, —CONH₂, —COOH, —(C₆-C₁₂)aryl;
R⁶ is independently selected from the group consisting of —(C₁-C₃₀)alkyl, —(C₂-C₁₂)alkenyl, —NH(C₁-C₁₀)alkyl, —NH(C₆-C₁₂)aryl, and

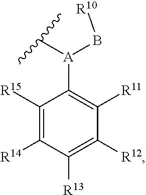

wherein in (C₁-C₃₀)alkyl, —(C₂-C₁₂)alkenyl, —NH(C₁-C₁₀)alkyl and —NH(C₆-C₁₂)aryl one or more hydrogen atoms are optionally independently substituted by a substituent selected from the group consisting of —OH, —O(C₁-C₁₀)alkyl, —(C₁-C₁₀)alkyl, —C(O)(C₁-C₁₀)alkyl, —COO(C₁-C₁₀)alkyl, —CONH₂, —COOH, (C₆-C₁₂)aryl;
A is N or CR¹⁶;
B is a bond, (C₁-C₈)alkylene, or (C₂-C₈)alkenylene;
R¹⁰ is selected from the group consisting of H, —COOH, —CONH₂, —COO(C₁-C₆)alkyl
R¹¹, R¹², R¹³, R¹⁴, R¹⁵ are independently selected from the group consisting of H, (C₁-C₄) alkyl, —O(C₁-C₄)alkyl, —OH, halogen, —CF₃, —CO(C₁-C₄)alkyl, COOH, —COO(C₁-C₆)alkyl, —CONH₂, —NH₂, —NHCO(C₁-C₄)alkyl, —NH(C₁-C₄)alkyl, —N((C₁-C₄)alkyl)₂; preferably H, —(C₁-C₄) alkyl, —O(C₁-C₄)alkyl, —OH, Cl, F, —CF₃, —CO(C₁-C₄)alkyl, COOH; more preferably H, —(C₁-C₄) alkyl, —O(C₁-C₄)alkyl, —OH, Cl, —CO(C₁-C₄)alkyl, COOH;
R¹⁶ is H, (C₁-C₄) alkyl, OH; preferably, H, CH₃ or OH.

The quinone of the present invention also comprises a pharmaceutically acceptable salt, solvate, enantiomer or hydrate thereof.

The invention further relates to a quinone according to formula (III)

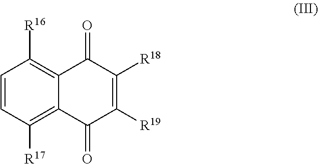

wherein
R¹⁶ and R¹⁷ are independently selected from the group consisting of H and —OH;
R¹⁸ is selected from the group consisting of H, —(C₁-C₃₀)alkyl, trifluoromethyl;
R¹⁹ is,

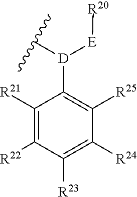

D is N;
E is a bond, (C₁-C₈)alkylene, or (C₂-C₈)alkenylene;
R²⁰ is selected from the group consisting of H, —COOH, —CONH₂, —COO(C₁-C₆)alkyl;
R²¹, R²², R²³, R²⁴, R²⁵ are independently selected from the group consisting of H, (C₁-C₄) alkyl, —O(C₁-C₄)alkyl, —OH, halogen, —CF₃, —CO(C₁-C₄)alkyl, COOH, —COO(C₁-C₆)alkyl, —CONH₂, —NH₂, —NHCO(C₁-C₄)alkyl, —NH(C₁-C₄)alkyl, and —N((C₁-C₄)alkyl)₂; preferably H, —(C₁-C₄) alkyl, —O(C₁-C₄)alkyl, —OH, Cl, F, —CF₃, —CO(C₁-C₄)alkyl, and COOH; more preferably H, —(C₁-C₄) alkyl, —O(C₁-C₄)alkyl, —OH, Cl, —CO(C₁-C₄)alkyl, and COOH;
wherein in (C₁-C₃₀)alkyl, one or more hydrogen atoms are optionally independently substituted by a substituent selected from the group consisting of —OH, —O(C₁-C₁₀)alkyl, (C₁-C₁₀)alkyl, —C(O)(C₁-C₁₀)alkyl, —COO(C₁-C₁₀)alkyl, —CONH₂, —COOH, —(C₆-C₁₂)aryl;
wherein the conditions i) and ii) are not present in the same molecule:
i) E is a bond, R²⁰ is H,
ii) R¹⁸ is H;
or a pharmaceutically acceptable salt, solvate, enantiomer, diastereomer, racemate or hydrate thereof.

### BRIEF DESCRIPTION OF THE FIGURE

FIG. 1: Seratrodast inhibits ferroptosis by suppressing lipid peroxidation. (A) Chemical structure of Seratrodast. (B) Seratrodast (Sera) dose-dependently suppresses ferroptosis induced by 100 nM RSL3 or 1.5 μM IKE for 18 h. Data plotted are mean±SD (n=4). (C) Seratrodast does not inhibit necroptosis. T+zV+L=20 ng/ml TNFα+10 μM Z-VAD-FMK+10 μM LCL161 for 18 h; 10 μM Necrostatin-1 (Nec-1); 6 μM Seratrodast; ****p≤0.0001 (1-way ANOVA test); data plotted are mean±SD (n=3). (D) Seratrodast does not inhibit apoptosis. 1 μM Staurosporine (Stauro); 50 μM Z-VAD-FMK; 6 μM Seratrodast for 18 h; ****p≤0.0001 (1-way ANOVA test); data plotted are mean±SD (n=3). (E) Seratrodast inhibits ferroptosis induced by RSL3, IKE, FINO2 and FIN56, comparable to Ferrostatin-1. 2 μM Fer-1; 6 μM Seratrodast for 18 h. Cell viability was normalized to DMSO-treated cells. Data plotted are mean±SEM (n=4). (F) Seratrodast inhibits ferroptosis in a 3D spheroid model. 200 nM RSL3; 2 μM Fer-1; 6 μM Seratrodast for 48 h. Representative image of n=8 spheroids per condition is shown; spheroid roundness was quantified (n=8); ****p≤0.0001 (1-way ANOVA test). (G) TXA2 receptor antagonists that lack a quinone moiety are not able to inhibit ferroptosis. 200 nM RSL3 and indicated antagonist concentration range for 18 h; data plotted are mean±SD (n=3). (H) Seratrodast displays antioxidant effects in a cell-free oxidizable BODIPY-C₁₁ assay treated with 7.5 mM free-radical-producing 2,2′-azobis(2-methyl-propanimidamide) dihydrochloride (AAPH). A significant decrease in oxidative fluorescence has been observed. 25 μM Fer-1; 25 μM Seratrodast; ****p≤0.0001 (1-way ANOVA test); data plotted are mean±SD (n=3). (1) Seratrodast reduces RSL3-induced malondialdehyde (MDA)—a product of lipid peroxidation. 250 nM RSL3; 2 μM Fer-1; 6 μM Seratrodast for 2.5 h. *p≤0.05 (1-way ANOVA test); data plotted are mean±SD (n=3). (J) Seratrodast limits RSL3-mediated oxidation of the lipid peroxidation sensor BODIPY-C11. 200 nM RSL3; 2 μM Fer-1; 6 μM Seratrodast for 2.5 h. Representative histogram is shown. Quantification of n=3 biological replicates is depicted; ****p≤0.0001 (1-way ANOVA test); data plotted are mean±SD (n=3). (K) Seratrodast reduces RSL3-induced 4-hydroxynonenal (4-HNE)—a product of lipid peroxidation. 300 nM RSL3; 2 μM Fer-1; 6 μM Seratrodast for 2 h. Representative histogram is shown. Quantification of n=3 replicates is depicted; ***p≤0.001 (1-way ANOVA test); data plotted are mean±SD (n=3).

### DETAILED DESCRIPTION OF THE INVENTION

The present invention provides small molecules with quinone moieties that have the potential to inhibit ferroptosis.

### Definitions

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “alkyl” refers to a monoradical of a saturated straight or branched hydrocarbon. Preferably, the alkyl group comprises from 1 to 30 carbon atoms, i.e., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 carbon atoms more preferably 1 to 10 carbon atoms, such as 1 to 6 or 1 to 4 carbon atoms. Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, isoheptyl, n-octyl, 2-ethyl-hexyl, n-nonyl, n-decyl, n-undecyl, n-dodecyl, and the like.

The term “alkylene” refers to a diradical of a saturated straight or branched hydrocarbon. Preferably, the alkylene comprises from 1 to 8 carbon atoms, i.e., 1, 2, 3, 4, 5, 6, 7, 8, carbon atoms, preferably 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylenisomers (e.g., 1,1-hexylene, 1,2-hexylene, 1,3-hexylene, 1,4-hexylene, 1,5-hexylene, 1,6-hexylene, and 1,1-isohexylene), and the like.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 4, i.e., 1, 2, 3, or 4, carbon-carbon double bonds. Preferably, the alkenyl group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkenyl group comprises from 2 to 10 carbon atoms and 1, 2, 3, 4, or 5 carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 2-methylpent-2-enyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, and the like. If an alkenyl group is attached to a nitrogen atom, the double bond cannot be alpha to the nitrogen atom.

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 4, i.e., 1, 2, 3, or 4, carbon-carbon double bonds. Preferably, the alkenylene group comprises from 2 to 10 carbon atoms, i.e., 2, 3, 4, 5, 6, 7, 8, 9, or 10 carbon atoms, more preferably 2 to 8 carbon atoms, such as 2 to 6 carbon atoms or 2 to 4 carbon atoms. Thus, in a preferred embodiment, the alkenylene group comprises from 2 to 10 carbon atoms and 1, 2, 3, 4, or 5 carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinyliden, 1-propen-1,2-diyl, 1-propen-1,3-diyl, 1-propen-2,3-diyl, allyliden, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl, 2-buten-2,3-diyl, 2-buten-2,4-diyl, 2-buten-3,4-diyl, and the like. If an alkenylene group is attached to a nitrogen atom, the double bond cannot be alpha to the nitrogen atom.

A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

“Isomers” are compounds having the same molecular formula but differ in structure (“structural isomers”) or in the geometrical positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (±).

“Diastereomers” are stereoisomers which are non-superimposable mirror-images of each other. “Tautomers” are structural isomers of the same chemical substance that spontaneously interconvert with each other, even when pure.

The term “solvate” as used herein refers to an addition complex of a dissolved material in a solvent (such as an organic solvent (e.g., an aliphatic alcohol (such as methanol, ethanol, n-propanol, isopropanol), acetone, acetonitrile, ether, and the like), water or a mixture of two or more of these liquids), wherein the addition complex exists in the form of a crystal or mixed crystal. The amount of solvent contained in the addition complex may be stoichiometric or nonstoichiometric. A “hydrate” is a solvate wherein the solvent is water.

### Compounds

The invention comprises a quinone, wherein the quinone is selected from a quinone as specified in one of the general formula (I) or (II)

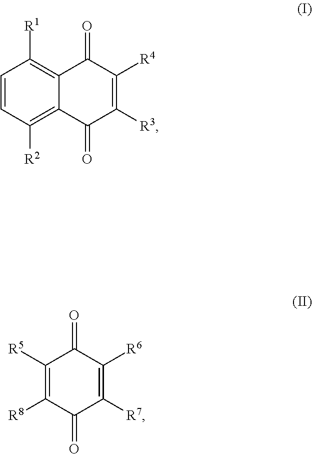

R¹ and R² are independently selected from the group consisting of H and —OH.

R³ is selected from the group consisting of H, —(C₁-C₃₀)alkyl, —(C₂-C₁₂)alkenyl, —OH, Cl, —O(C₁-C₁₀)alkyl, trifluoromethyl.

Preferably R³ is selected from the group consisting of H, —(C₁-C₅)alkyl, —O(C₁-C₅)alkyl, —OH, trifluoromethyl, and Cl.

More preferably, R³ is selected from the group consisting of H, —CH₃, —OH, —OMe, trifluoromethyl and Cl.

Most preferably R³ is selected from group consisting of H, —CH₃, —OH, trifluoromethyl, and Cl.

R⁴ is independently selected from the group consisting of —(C₁-C₃₀)alkyl, —(C₂-C₁₂)alkenyl, —NH(C₁-C₁₀)alkyl, —NH(C₆-C₁₂)aryl, and

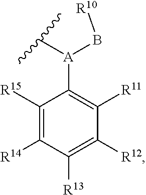

wherein in (C₁-C₃₀)alkyl, —(C₂-C₁₂)alkenyl, —NH(C₁-C₁₀)alkyl and —NH(C₆-C₁₂)aryl one or more hydrogen atoms are optionally independently substituted by a substituent selected from the group consisting of —OH, —O(C₁-C₁₀)alkyl, (C₁-C₁₀)alkyl, —C(O)(C₁-C₁₀)alkyl, —COO(C₁-C₁₀)alkyl, —CONH₂, —(C₆-C₁₀)aryl, —COOH, (C₆-C₁₂)aryl.

R⁵ is selected from the group consisting of H, —(C₁-C₃₀)alkyl, —(C₂-C₁₂)alkenyl, —OH, Cl, and —O(C₁-C₁₀)alkyl, trifluoromethyl.

Preferably, R⁵ is selected from the group consisting of H, —(C₁-C₁₀)alkyl, —O(C₁-C₁₀)alkyl.

More preferably, R⁵ is selected from the group consisting of H, —CH₃, —OCH₃.

Most preferably, R⁵ is selected from the group consisting of —CH₃, —OCH₃.

R⁶ is independently selected from the group consisting of —(C₁-C₃₀)alkyl, —(C₂-C₁₂)alkenyl, —NH(C₁-C₁₀)alkyl, —NH(C₆-C₁₂)aryl, and

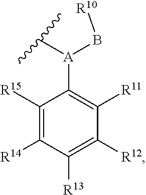

wherein in (C₁-C₃₀)alkyl, —(C₂-C₁₂)alkenyl, —NH(C₁-C₁₀)alkyl and —NH(C₆-C₁₂)aryl one or more hydrogen atoms are optionally independently substituted by a substituent selected from the group consisting of —OH, —O(C₁-C₁₀)alkyl, —(C₁-C₁₀)alkyl, —C(O)(C₁-C₁₀)alkyl, —COO(C₁-C₁₀)alkyl, —CONH₂, —(C₅-C₁₀)aryl, —COOH, (C₆-C₁₂)aryl.

R⁷ is selected from the group consisting of H, —(C₁-C₃₀)alkyl, —(C₂-C₁₂)alkenyl, —OH, —Cl, —O(C₁-C₁₀)alkyl, trifluoromethyl.

Preferably, R⁷ is selected from the group consisting of H, —(C₁-C₅)alkyl, —O(C₁-C₅)alkyl, —OH, and —Cl.

More preferably, R⁷ is selected from the group consisting of H, —CH₃, —OH, —OMe and Cl.

Most Preferably R⁷ is selected from group consisting of H, —CH₃, —OH, and Cl.

R⁸ is selected from the group consisting of H, —(C₁-C₃₀)alkyl, —(C₂-C₁₂)alkenyl, —OH, —Cl, —O(C₁-C₁₀)alkyl, trifluoromethyl.

Preferably, R⁸ is selected from the group consisting of H, —(C₁-C₁₀)alkyl, —O(C₁-C₁₀)alkyl.

More preferably, R⁸ is selected from the group consisting of H, —CH₃, —OCH₃.

Most preferably, R⁸ is selected from the group consisting of —CH₃, —OCH₃.

A is N or CR¹⁶.

B is a bond, (C₁-C₈)alkylene, or (C₂-C₈)alkenylene.

R¹⁰ is selected from the group consisting of H, —COOH, —CONH₂, —COO(C₁-C₆)alkyl

R¹¹, R¹², R¹³, R¹⁴, R¹⁵ are independently selected from the group consisting of H, (C₁-C₄) alkyl, —O(C₁-C₄)alkyl, —OH, halogen, —CF₃, —CO(C₁-C₄)alkyl, COOH, —COO(C₁-C₆)alkyl, —CONH₂, —NH₂, —NHCO(C₁-C₄)alkyl, —NH(C₁-C₄)alkyl, —N((C₁-C₄)alkyl)₂; preferably H, —(C₁-C₄) alkyl, —OCH₃, —OH, Cl, F, —CF₃, —CO(C₁-C₄)alkyl, COOH; more preferably H, —(C₁-C₄) alkyl, —OCH₃, —OH, Cl, F, —CO(C₁-C₄)alkyl, COOH.

In one embodiment R¹³ is selected from the group consisting of —C(O)CH₃, -Ethyl, -Methyl, —OH, —OCH₃, and R¹¹, R¹², R¹⁴, R¹⁵ are H.

In one embodiment R¹² is —C(O)CH₃, and R¹¹, R¹³, R¹⁴, R¹⁵ are H.

In one embodiment R¹¹ and R¹³ are Methyl and R¹², R¹⁴, R¹⁵ are H.

R¹⁶ is H, (C₁-C₄) alkyl, OH; preferably, H, CH₃ or OH.

In one embodiment, the quinone is a quinone according to formula (III)

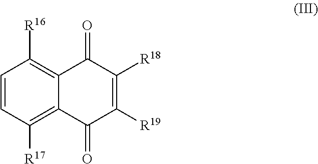

wherein
R¹⁶ and R¹⁷ are independently selected from the group consisting of H and —OH; preferably H;
R¹⁸ is selected from the group consisting of H, —(C₁-C₃₀)alkyl, trifluoromethyl; preferably H, —CH₃, trifluoromethyl;
R¹⁹ is,

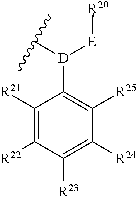

D is N;
E is a bond, (C₁-C₈)alkylene, or (C₂-C₈)alkenylene, preferably a bond, (C₁-C₈)alkylene; more preferably (C₃-C₅)alkylene;
R²⁰ is selected from the group consisting of H, —COOH, —CONH₂, —COO(C₁-C₆)alkyl; preferably H, —COOH, —CONH₂;
R²¹, R²², R²³, R²⁴, R²⁵ are independently selected from the group consisting of H, (C₁-C₄) alkyl, —O(C₁-C₄)alkyl, —OH, halogen, —CF₃, —CO(C₁-C₄)alkyl, COOH, —COO(C₁-C₆)alkyl, —CONH₂, and —N((C₁-C₄)alkyl)₂; preferably H, —(C₁-C₄) alkyl, —O(C₁-C₄)alkyl, —OH, Cl, F, —CF₃, —CO(C₁-C₄)alkyl, and COOH; more preferably H, —(C₁-C₄) alkyl, —O(C₁-C₄)alkyl, —OH, Cl, —CO(C₁-C₄)alkyl, and COOH; particular preferred H, (C₁-C₄) alkyl, —NHCO(C₁-C₄)alkyl;
wherein in (C₁-C₃₀)alkyl, one or more hydrogen atoms are optionally independently substituted by a substituent selected from the group consisting of —OH, —O(C₁-C₁₀)alkyl, (C₁-C₁₀)alkyl, —C(O)(C₁-C₁₀)alkyl, —COO(C₁-C₁₀)alkyl, —CONH₂, —COOH, —(C₆-C₁₂)aryl;
wherein the conditions i) and ii) are not present in the same molecule:
i) E is a bond, R²⁰ is H,
ii) R¹⁸ is H;
or a pharmaceutically acceptable salt, solvate, enantiomer, diastereomer, racemate or hydrate thereof.

In particular, the quinone is selected from the quinones of Table 1.

TABLE 1

| | |
|---|---|
| 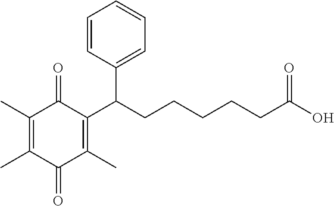 [SMILES: CC1=C(C)C(=O)C(C(CCCCCC(=O)O)c2ccccc2)=C(C)C1=O] | 1 |
| 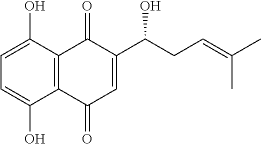 [SMILES: CC(C)=CC[C@@H](O)C1=CC(=O)c2c(O)ccc(O)c2C1=O] | 2 |
| 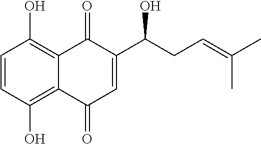 [SMILES: CC(C)=CC[C@H](O)C1=CC(=O)c2c(O)ccc(O)c2C1=O] | 3 |
| 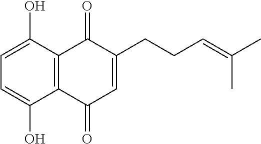 [SMILES: CC(C)=CCCC1=CC(=O)c2c(O)ccc(O)c2C1=O] | 4 |
| 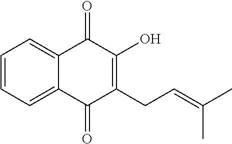 [SMILES: CC(C)=CCC1=C(O)C(=O)c2ccccc2C1=O] | 5 |
| 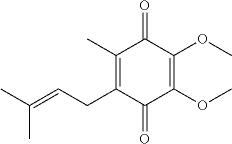 [SMILES: COC1=C(OC)C(=O)C(CC=C(C)C)=C(C)C1=O] | 6 |
| 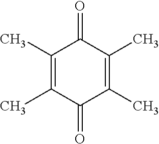 | 7 |
| 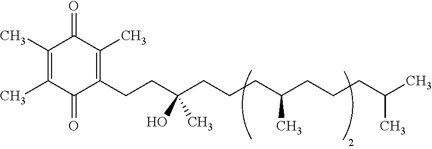 | 8 |
| 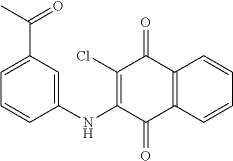 [SMILES: CC(=O)c1cccc(NC2=C(Cl)C(=O)c3ccccc3C2=O)c1] | 9 |
| 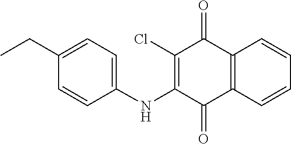 [SMILES: CCc1ccc(NC2=C(Cl)C(=O)c3ccccc3C2=O)cc1] | 10 |
| 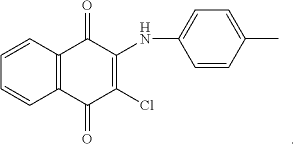 [SMILES: Cc1ccc(NC2=C(Cl)C(=O)c3ccccc3C2=O)cc1] | 11 |
| 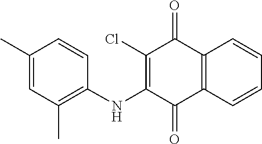 [SMILES: Cc1ccc(NC2=C(Cl)C(=O)c3ccccc3C2=O)c(C)c1] | 12 |
| 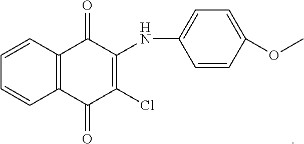 [SMILES: COc1ccc(NC2=C(Cl)C(=O)c3ccccc3C2=O)cc1] | 13 |
| 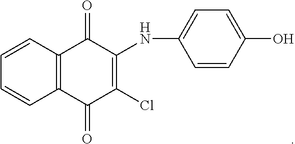 [SMILES: O=C1C(Cl)=C(Nc2ccc(O)cc2)C(=O)c2ccccc21] | 14 |
| 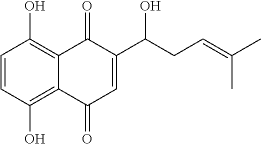 [SMILES: CC(C)=CCC(O)C1=CC(=O)c2c(O)ccc(O)c2C1=O] | 15 |
| 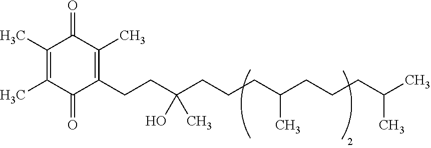 | 16 |
| 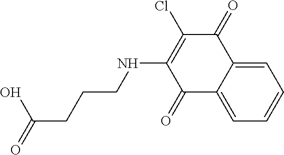 [SMILES: O=C(O)CCCNC1=C(Cl)C(=O)c2ccccc2C1=O] | 17 |
| 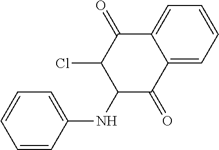 [SMILES: O=C1c2ccccc2C(=O)C(Nc2ccccc2)C1Cl] | 18 |
| 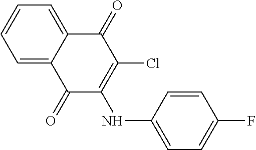 [SMILES: O=C1C(Cl)=C(Nc2ccc(F)cc2)C(=O)c2ccccc21] | 19 |
| 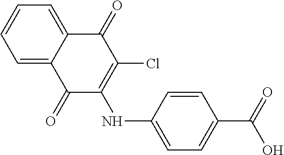 [SMILES: O=C(O)c1ccc(NC2=C(Cl)C(=O)c3ccccc3C2=O)cc1] | 20 |
| 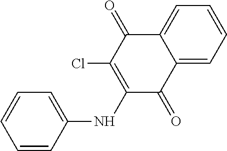 [SMILES: O=C1C(Cl)=C(Nc2ccccc2)C(=O)c2ccccc21] | 21 |
| 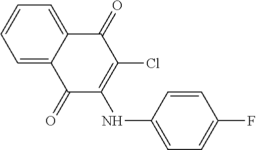 [SMILES: O=C1C(Cl)=C(Nc2ccc(F)cc2)C(=O)c2ccccc21] | 22 |
| 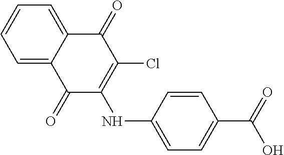 [SMILES: O=C(O)c1ccc(NC2=C(Cl)C(=O)c3ccccc3C2=O)cc1] | 23 |
| 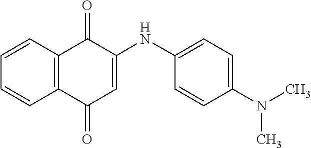 [SMILES: CN(C)c1ccc(NC2=CC(=O)c3ccccc3C2=O)cc1] | 24 |
| 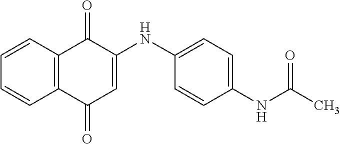 [SMILES: CC(=O)Nc1ccc(NC2=CC(=O)c3ccccc3C2=O)cc1] | 25 |
| 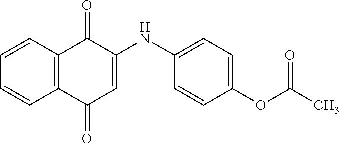 [SMILES: CC(=O)Oc1ccc(NC2=CC(=O)c3ccccc3C2=O)cc1] | 26 |
| 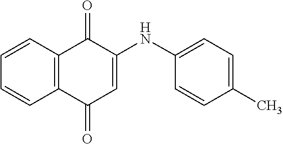 [SMILES: Cc1ccc(NC2=CC(=O)c3ccccc3C2=O)cc1] | 27 |
| 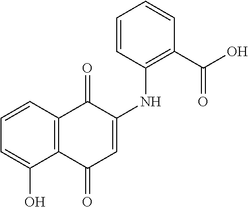 [SMILES: O=C(O)c1ccccc1NC1=CC(=O)c2c(O)cccc2C1=O] | 28 |
| 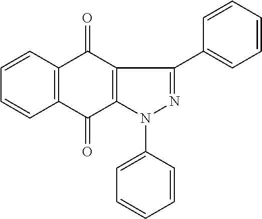 [SMILES: O=C1c2ccccc2C(=O)c2c1c(-c1ccccc1)nn2-c1ccccc1] | 29 |
| 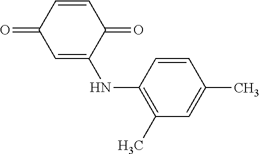 [SMILES: Cc1ccc(NC2=CC(=O)C=CC2=O)c(C)c1] | 30 |
| 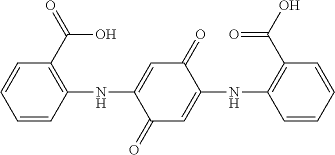 [SMILES: O=C1C=C(Nc2ccccc2C(=O)O)C(=O)C=C1Nc1ccccc1C(=O)O] | 31 |
| 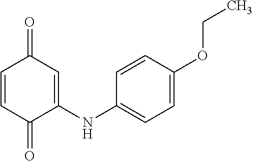 [SMILES: CCOc1ccc(NC2=CC(=O)C=CC2=O)cc1] | 32 |
| 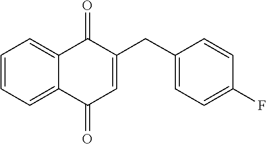 [SMILES: O=C1C=C(Cc2ccc(F)cc2)C(=O)c2ccccc21] | 33 |
| 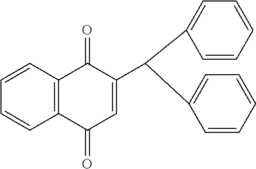 [SMILES: O=C1C=C(C(c2ccccc2)c2ccccc2)C(=O)c2ccccc21] | 34 |
| 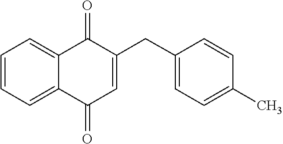 [SMILES: Cc1ccc(CC2=CC(=O)c3ccccc3C2=O)cc1] | 35 |
| 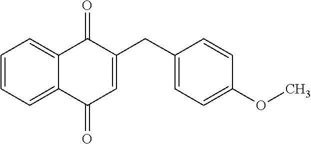 [SMILES: COc1ccc(CC2=CC(=O)c3ccccc3C2=O)cc1] | 36 |
| 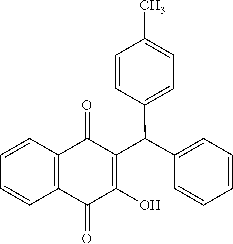 [SMILES: Cc1ccc(C(C2=C(O)C(=O)c3ccccc3C2=O)c2ccccc2)cc1] | 37 |
| 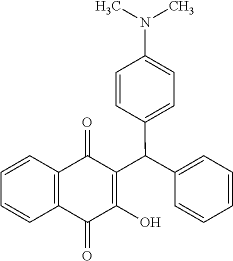 [SMILES: CN(C)c1ccc(C(C2=C(O)C(=O)c3ccccc3C2=O)c2ccccc2)cc1] | 38 |
| 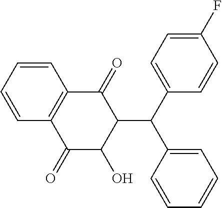 [SMILES: O=C1c2ccccc2C(=O)C(C(c2ccccc2)c2ccc(F)cc2)C1O] | 39 |
| 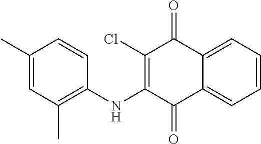 [SMILES: Cc1ccc(NC2=C(Cl)C(=O)c3ccccc3C2=O)c(C)c1] | 40 |
| 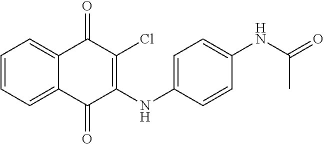 [SMILES: CC(=O)Nc1ccc(NC2=C(Cl)C(=O)c3ccccc3C2=O)cc1] | 41 |
| 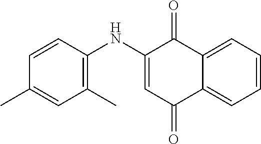 [SMILES: Cc1ccc(NC2=CC(=O)c3ccccc3C2=O)c(C)c1] | 42 |
| 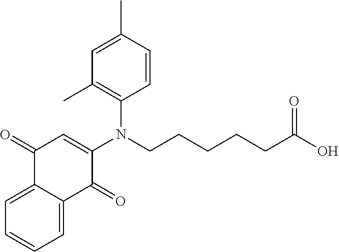 [SMILES: Cc1ccc(N(CCCCCC(=O)O)C2=CC(=O)c3ccccc3C2=O)c(C)c1] | 43 |
| 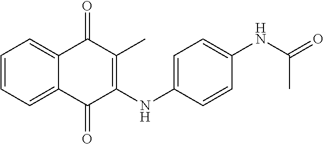 [SMILES: CC(=O)Nc1ccc(NC2=C(C)C(=O)c3ccccc3C2=O)cc1] | 44 |
| 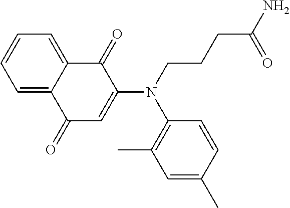 [SMILES: Cc1ccc(N(CCCC(N)=O)C2=CC(=O)c3ccccc3C2=O)c(C)c1] | 45 |
| 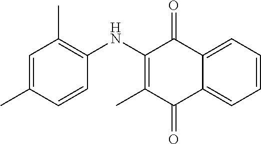 [SMILES: CC1=C(Nc2ccc(C)cc2C)C(=O)c2ccccc2C1=O] | 46 |
| 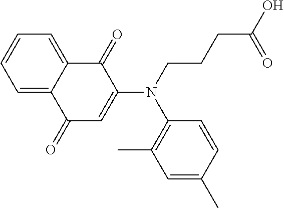 [SMILES: Cc1ccc(N(CCCC(=O)O)C2=CC(=O)c3ccccc3C2=O)c(C)c1] | 47 |
| 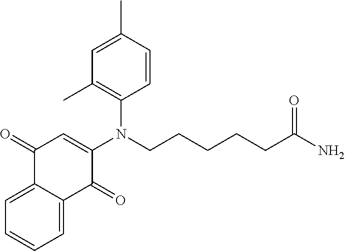 [SMILES: Cc1ccc(N(CCCCCC(N)=O)C2=CC(=O)c3ccccc3C2=O)c(C)c1] | 48 |
| 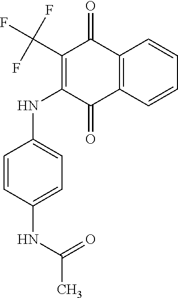 [SMILES: CC(=O)Nc1ccc(NC2=C(C(F)(F)F)C(=O)c3ccccc3C2=O)cc1] | 49 |
| 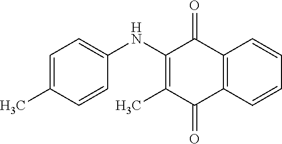 [SMILES: CC1=C(Nc2ccc(C)cc2)C(=O)c2ccccc2C1=O] | 50 |
| 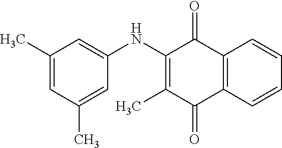 [SMILES: CC1=C(Nc2cc(C)cc(C)c2)C(=O)c2ccccc2C1=O] | 51 |
| 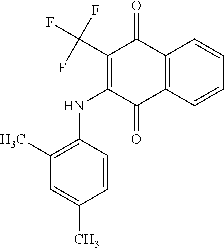 [SMILES: Cc1ccc(NC2=C(C(F)(F)F)C(=O)c3ccccc3C2=O)c(C)c1] | 52 |
| 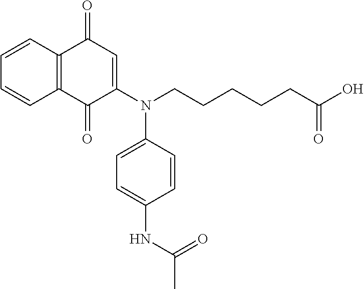 [SMILES: CC(=O)Nc1ccc(N(CCCCCC(=O)O)C2=CC(=O)c3ccccc3C2=O)cc1] | 53 |
| 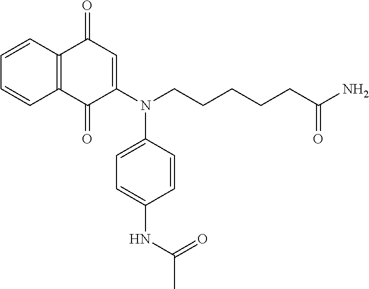 [SMILES: CC(=O)Nc1ccc(N(CCCCCC(N)=O)C2=CC(=O)c3ccccc3C2=O)cc1] | 54 |

In one embodiment, the quinone is selected from the group consisting of

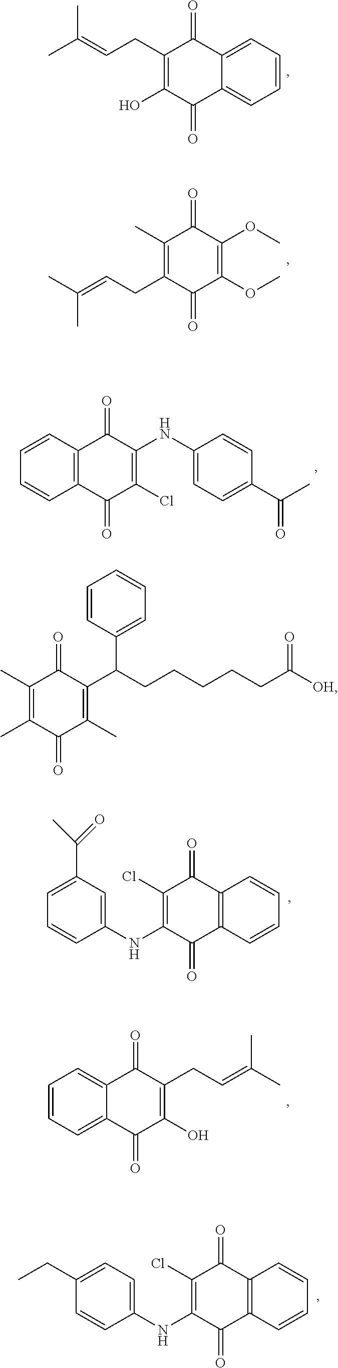

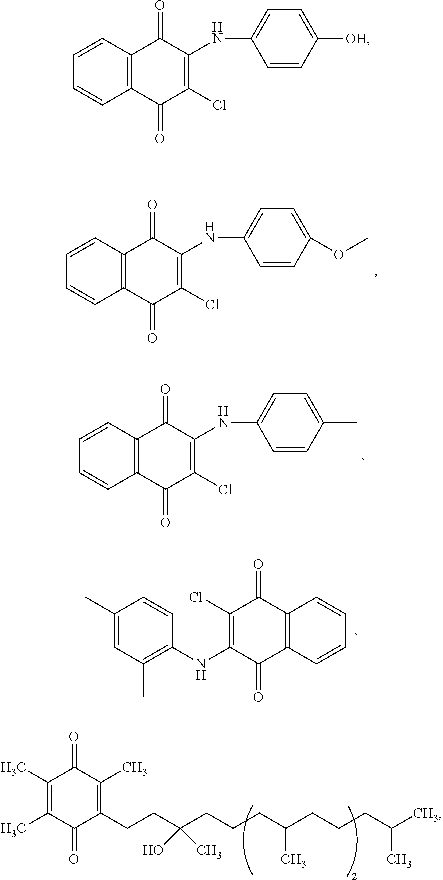

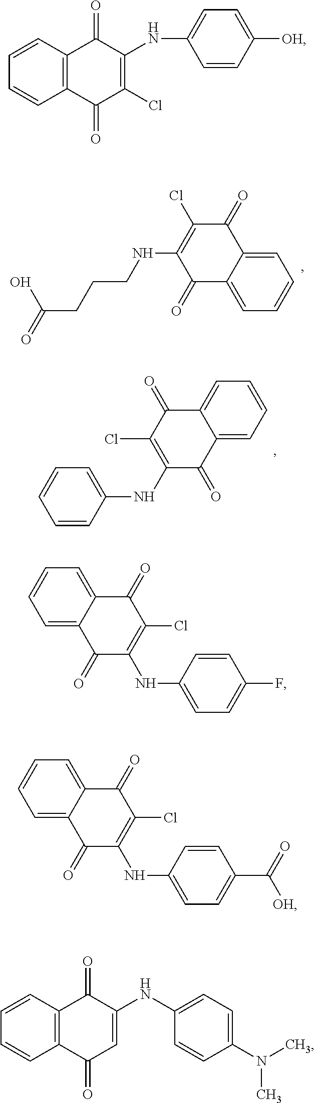

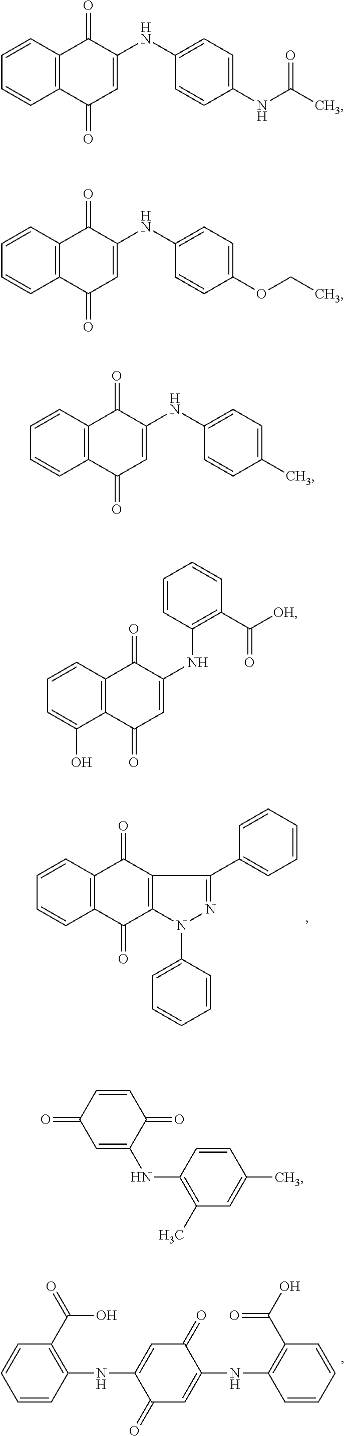

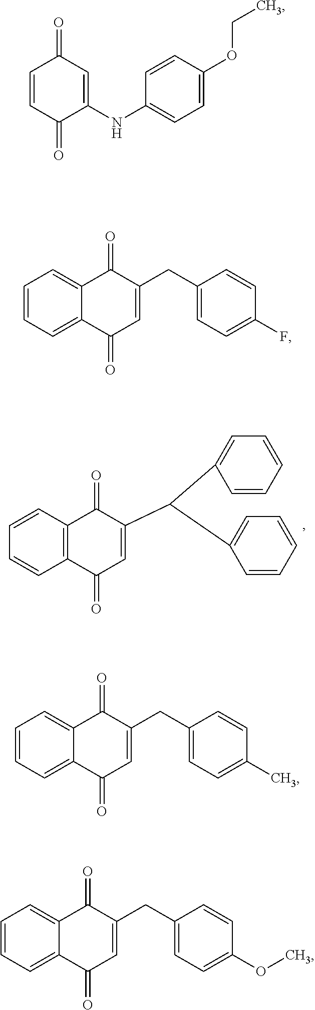 [SMILES: CCOc1ccc(NC2=CC(=O)C=CC2=O)cc1 ; O=C1C=C(Cc2ccc(F)cc2)C(=O)c2ccccc21 ; O=C1C=C(C(c2ccccc2)c2ccccc2)C(=O)c2ccccc21 ; Cc1ccc(CC2=CC(=O)c3ccccc3C2=O)cc1 ; COc1ccc(CC2=CC(=O)c3ccccc3C2=O)cc1]

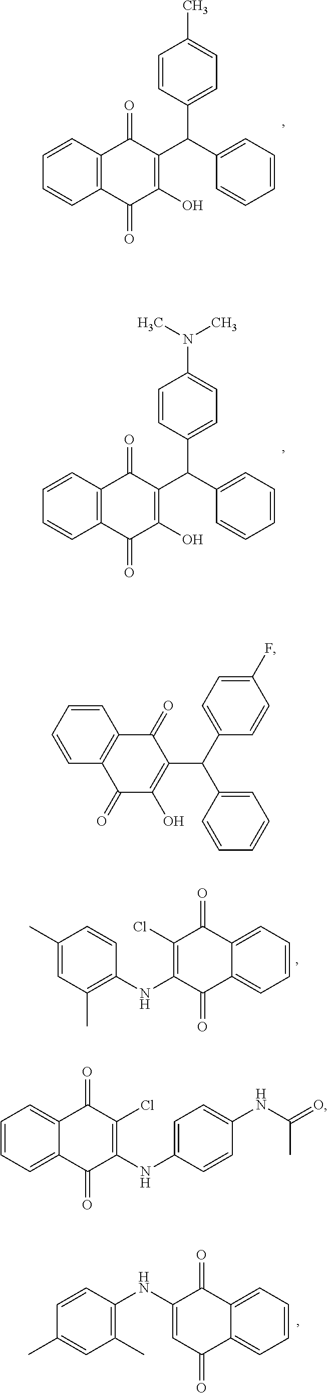

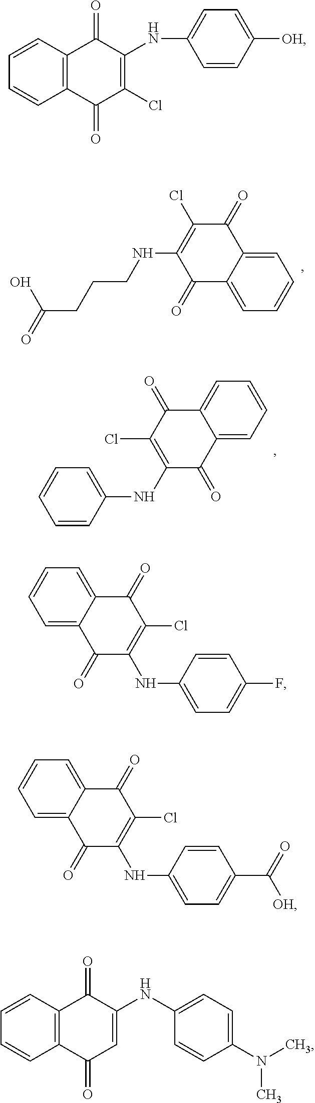

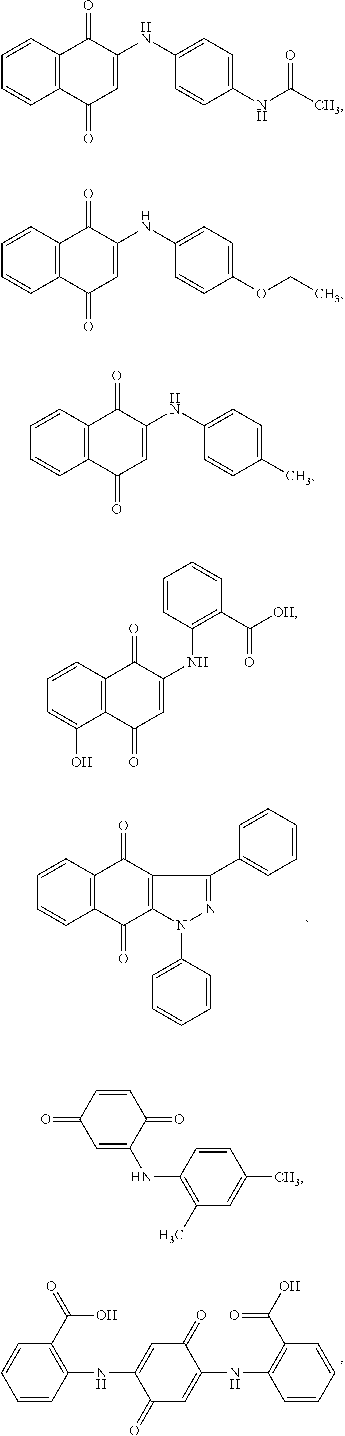

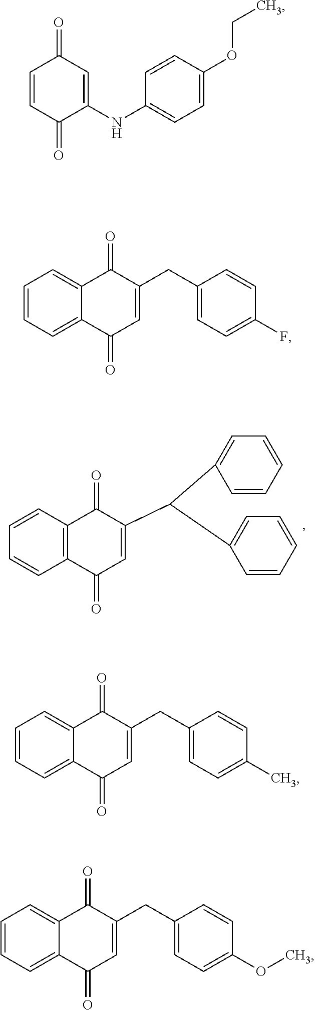 [SMILES: CCOc1ccc(NC2=CC(=O)C=CC2=O)cc1 ; O=C1C=C(Cc2ccc(F)cc2)C(=O)c2ccccc21 ; O=C1C=C(C(c2ccccc2)c2ccccc2)C(=O)c2ccccc21 ; Cc1ccc(CC2=CC(=O)c3ccccc3C2=O)cc1 ; COc1ccc(CC2=CC(=O)c3ccccc3C2=O)cc1]

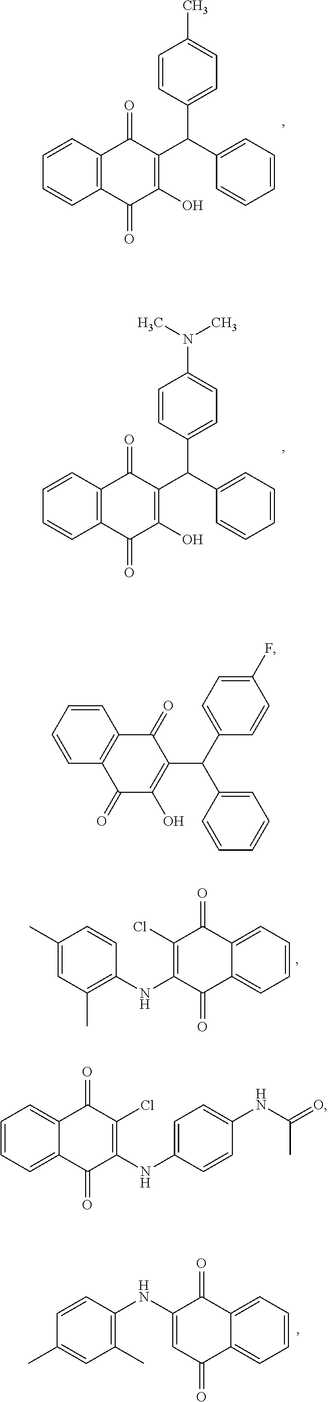

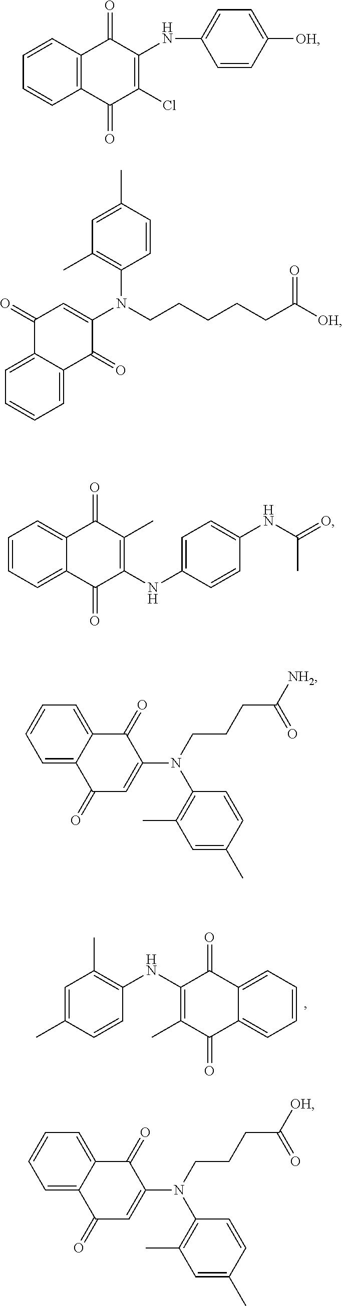

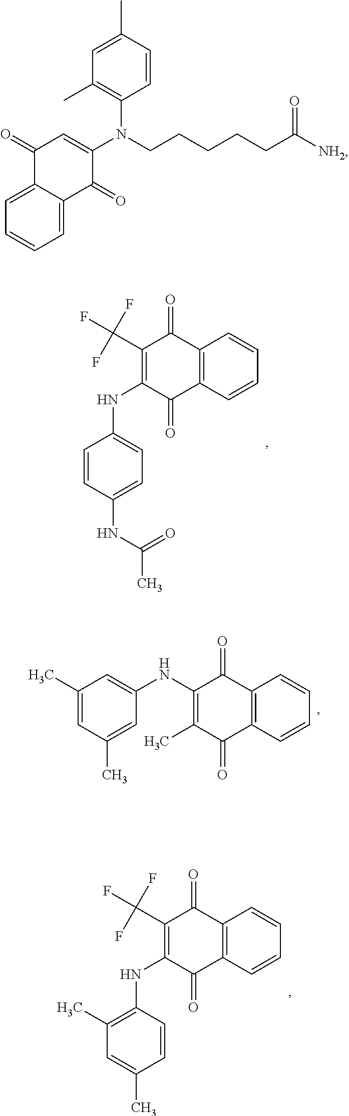 [SMILES: Cc1ccc(N(CCCCCC(N)=O)C2=CC(=O)c3ccccc3C2=O)c(C)c1 ; CC(=O)Nc1ccc(NC2=C(C(F)(F)F)C(=O)c3ccccc3C2=O)cc1 ; CC1=C(Nc2cc(C)cc(C)c2)C(=O)c2ccccc2C1=O ; Cc1ccc(NC2=C(C(F)(F)F)C(=O)c3ccccc3C2=O)c(C)c1]

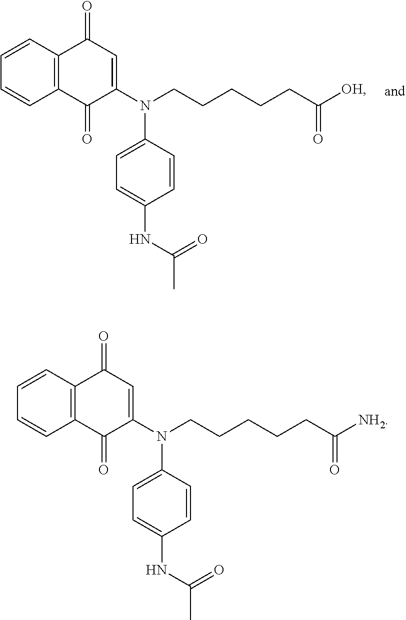 [SMILES: CC(=O)Nc1ccc(N(CCCCCC(=O)O)C2=CC(=O)c3ccccc3C2=O)cc1 ; CC(=O)Nc1ccc(N(CCCCCC(N)=O)C2=CC(=O)c3ccccc3C2=O)cc1]

In a further embodiment, the quinone is selected from the group consisting of

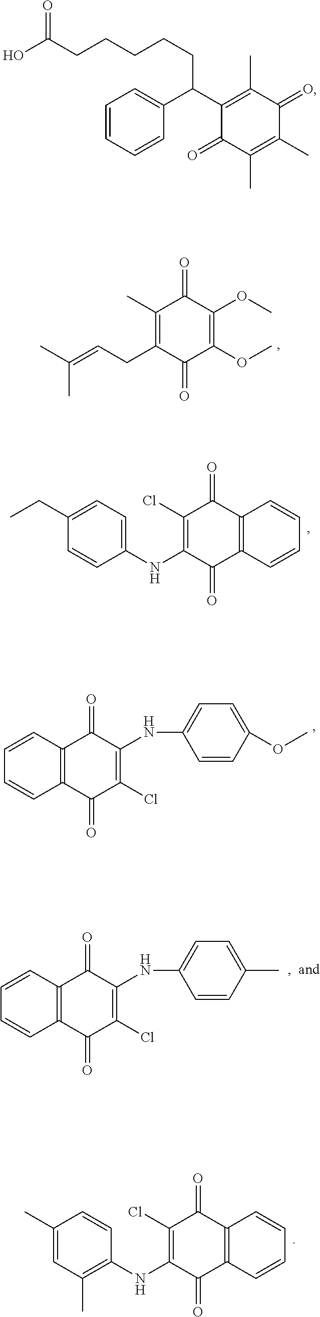

In a further embodiment, the quinone is selected from the quinone according to formula (I), (II), wherein the quinone according to formula (II) is selected from the group consisting of

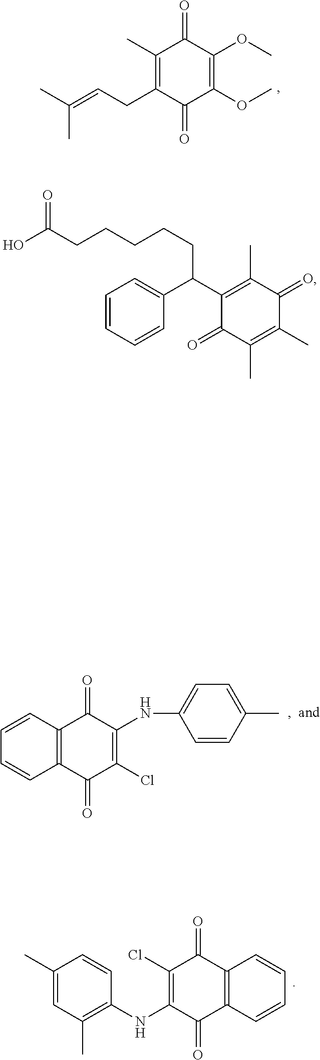

In one embodiment, the quinone is at least not one of the following:

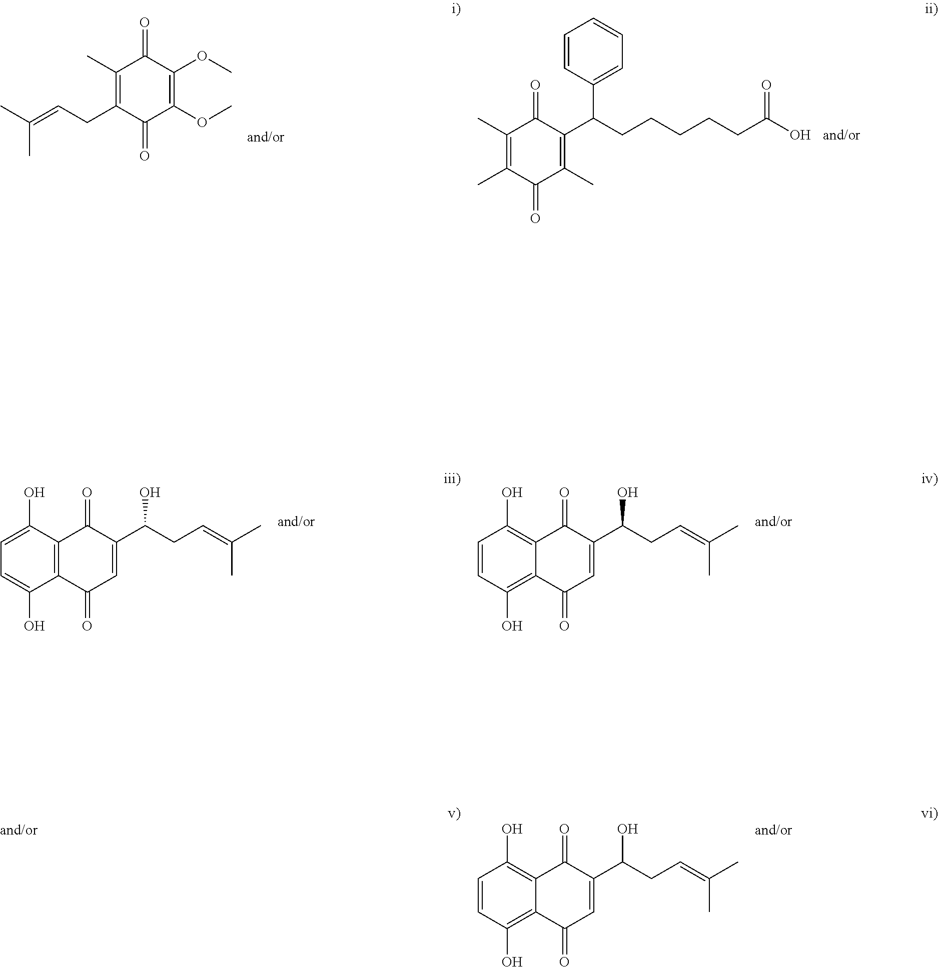 [SMILES: COC1=C(OC)C(=O)C(CC=C(C)C)=C(C)C1=O ; CC1=C(C)C(=O)C(C(CCCCCC(=O)O)c2ccccc2)=C(C)C1=O ; CC(C)=CC[C@@H](O)C1=CC(=O)c2c(O)ccc(O)c2C1=O ; CC(C)=CC[C@H](O)C1=CC(=O)c2c(O)ccc(O)c2C1=O ; CC(C)=CCC(O)C1=CC(=O)c2c(O)ccc(O)c2C1=O]

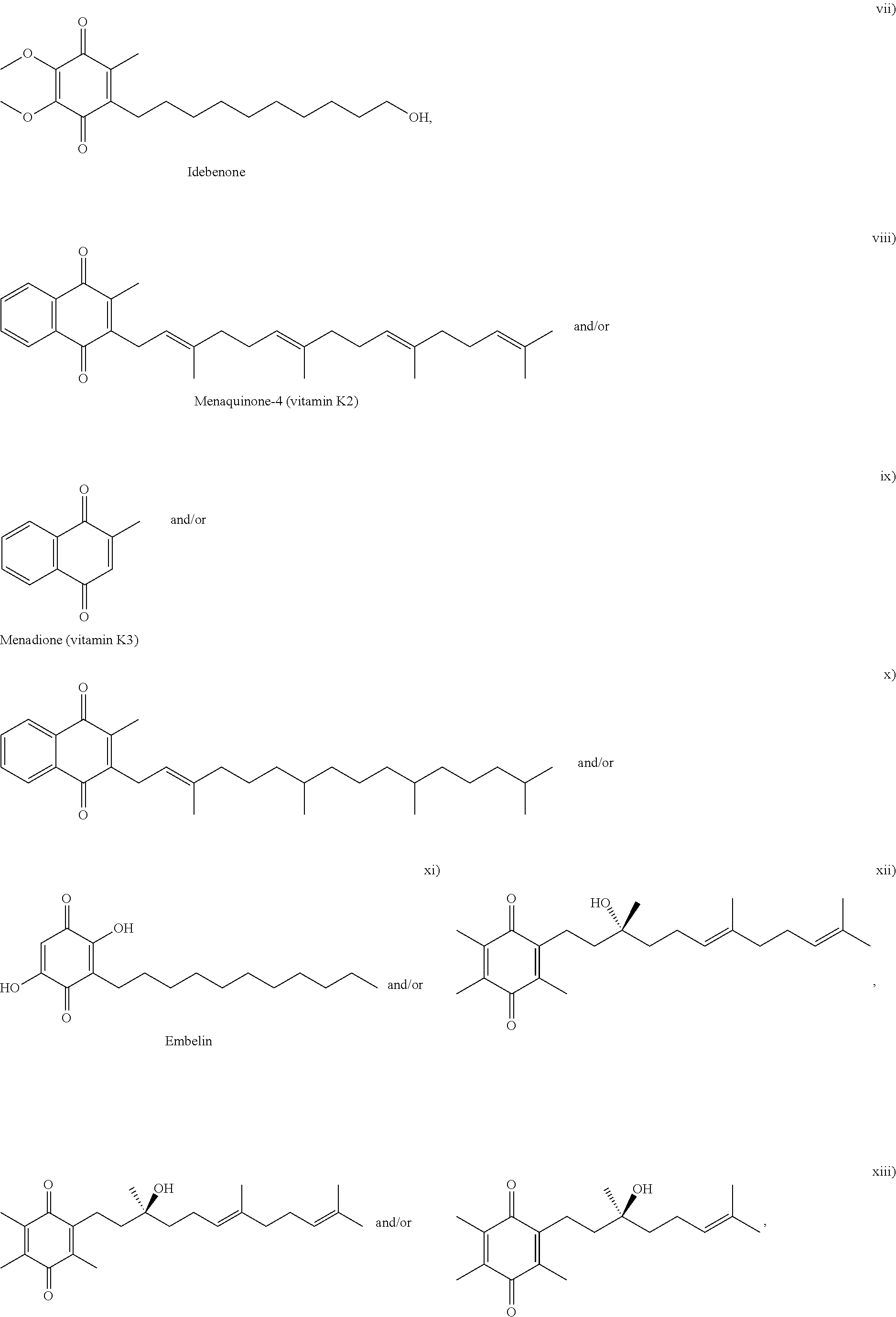

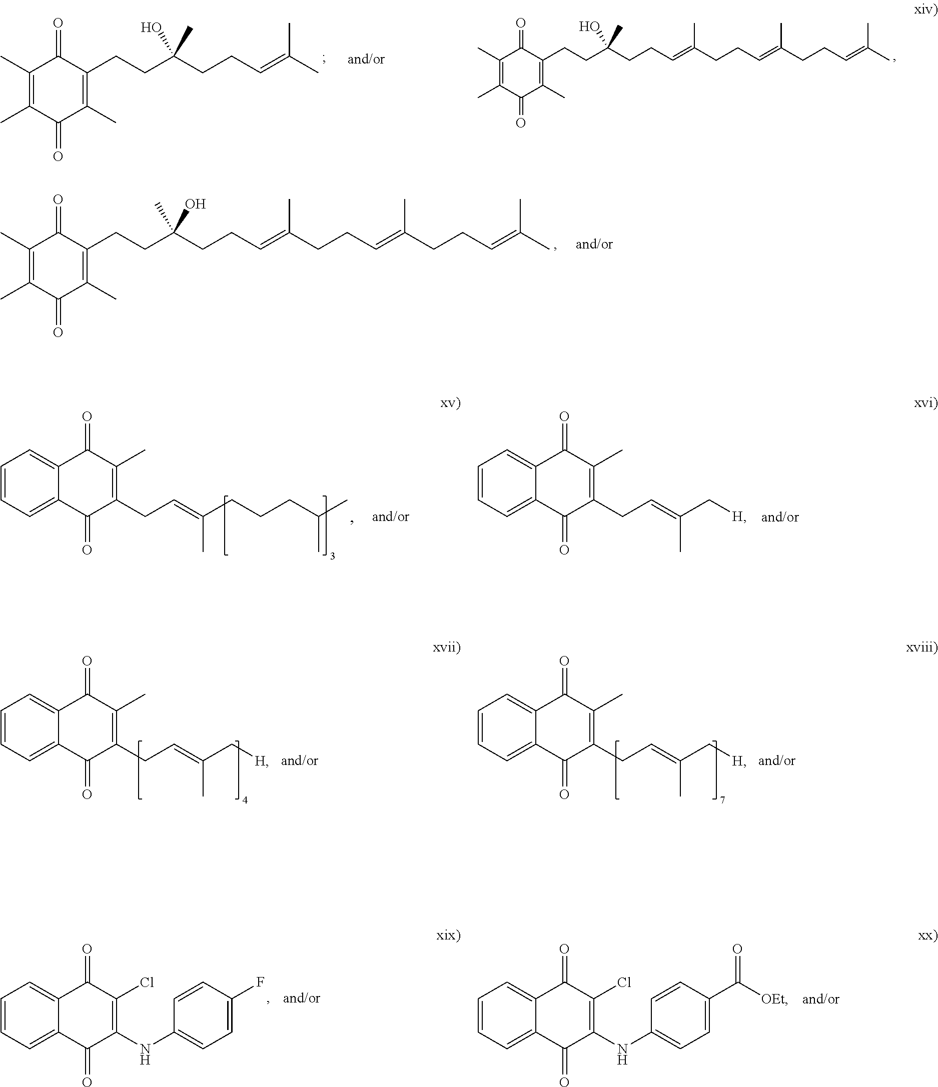

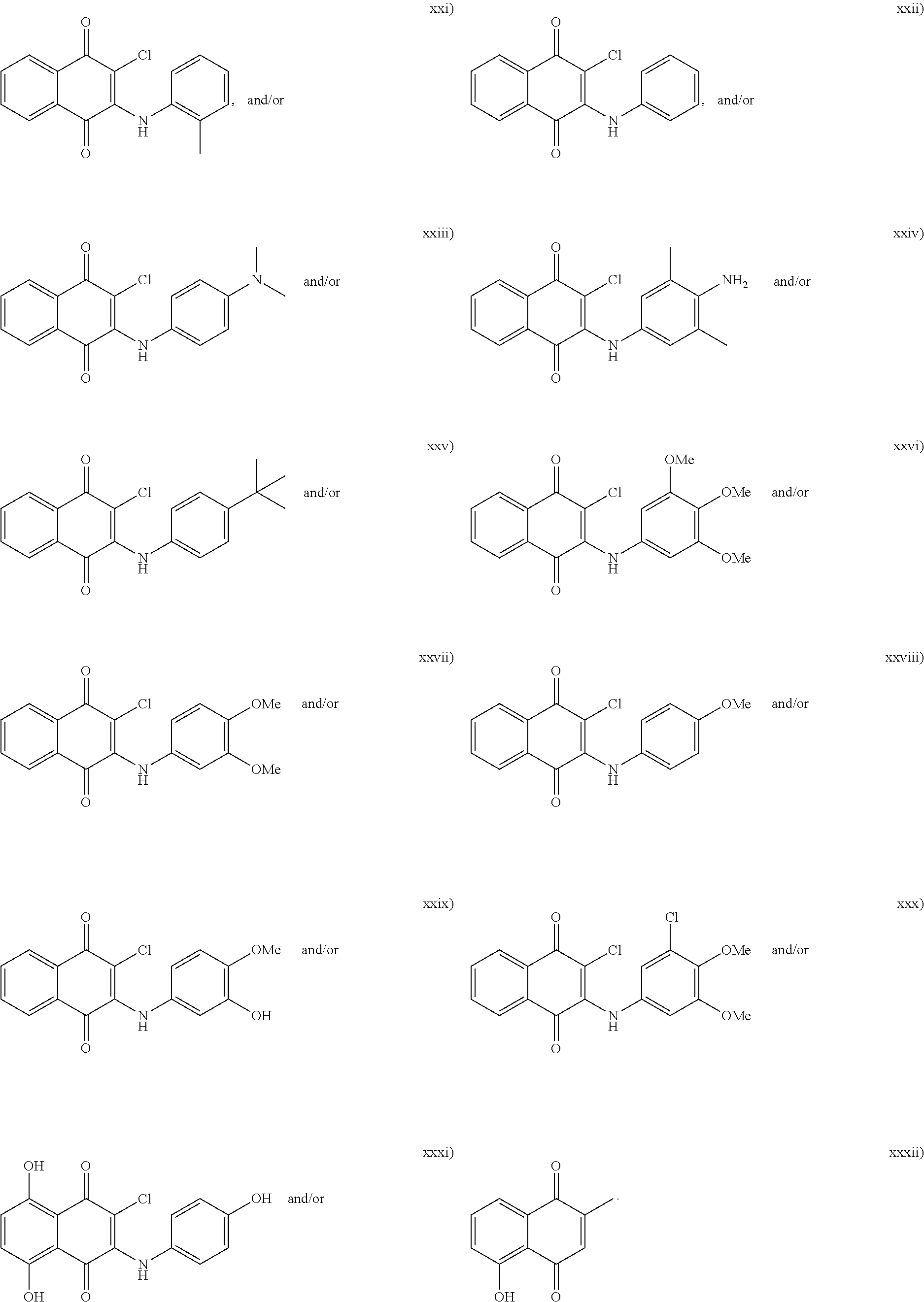

The quinone of the present invention comprises also a pharmaceutically acceptable salt, solvate, enantiomer, diastereomer, racemate or hydrate thereof.

Some of the inventive compounds may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% free of other enantiomers or diastereomers of the compounds, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

### Pharmaceutical Applications

The quinone of the present invention is for use in the inhibition of ferroptosis.

In particular, the quinone of the present invention is for use in the treatment of a disease which can be treated or prevented by inhibition of ferroptosis.

Preferably, the disease is selected from the group consisting of Amyotrophic Lateral Sclerosis (ALS); Alzheimer's Disease (AD); Parkinson's Disease (PD); Huntington's Disease (HD); COPD; ischemia reperfusion injury (IRI) of liver, kidney, intestine, lung; myocardial infarction; cardiomyopathy; stroke; traumatic brain injury; kidney degeneration; retinal degeneration (2, 10, 11).

Stockwell et al. (2) (FIG. 3, as well as on page 2411 ff.), Conrad et. al (10) and Hadian et al. (11) summarize that ferroptosis is associated with several diseases like Amyotrophic Lateral Sclerosis (ALS); Alzheimer's Disease (AD); Parkinson's Disease (PD); Huntington's Disease (HD); COPD; ischemia reperfusion injury (IRI) of liver, kidney, intestine, lung; myocardial infarction; cardiomyopathy; stroke; traumatic brain injury; kidney degeneration; retinal degeneration.

The invention comprises a pharmaceutical composition comprising the quinone for use as defined above and a pharmaceutically acceptable carrier.

“Pharmaceutical composition” refers to one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

“Pharmaceutically acceptable carrier” refers to a diluent, adjuvant, excipient, or vehicle with which the therapeutic is administered. Such pharmaceutically acceptable carrier can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid pharmaceutically acceptable carrier for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and pharmaceutically acceptable carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutically acceptable carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art (cf., e.g., Remington, “The Science and Practice of Pharmacy” edited by Allen, Loyd V., Jr., 22^{nd} edition, Pharmaceutical Sciences, September 2012; Ansel et al., “Pharmaceutical Dosage Forms and Drug Delivery Systems”, 7^{th} edition, Lippincott Williams & Wilkins Publishers, 1999.).

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound which is the lowest dose effective to produce a therapeutic effect. Such an effective dose will generally depend upon the factors described above. It is preferred that administration be oral, intravenous, intramuscular, intraperitoneal, or subcutaneous, preferably administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation/composition.

Generally, out of 100% (for the pharmaceutical formulations/compositions), the amount of active ingredient (in particular, the amount of the compound of the present invention, optionally together with other therapeutically active agents, if present in the pharmaceutical formulations/compositions) will range from about 0.01% to about 99%, preferably from about 0.1% to about 70%, most preferably from about 1% to about 30%, wherein the reminder is preferably composed of the one or more pharmaceutically acceptable excipients.

The amount of active ingredient, e.g., a compound of the invention, in a unit dosage form and/or when administered to an individual or used in therapy, may range from about 0.1 mg to about 1000 mg (for example, from about 1 mg to about 500 mg, such as from about 10 mg to about 200 mg) per unit, administration or therapy. In certain embodiments, a suitable amount of such active ingredient may be calculated using the mass or body surface area of the individual, including amounts of between about 1 mg/Kg and 10 mg/Kg (such as between about 2 mg/Kg and 5 mg/Kg), or between about 1 mg/m² and about 400 mg/m² (such as between about 3 mg/m² and about 350 mg/m² or between about 10 mg/m² and about 200 mg/m²).

In one embodiment, the compounds or compositions of the invention may be administered by infusion, preferably slow continuous infusion over a long period, such as more than 24 hours, in order to reduce toxic side effects. The administration may also be performed by continuous infusion over a period of from 2 to 24 hours, such as of from 2 to 12 hours. Such regimen may be repeated one or more times as necessary, for example, after 6 months or 12 months.

In yet another embodiment, the compounds or compositions of the invention are administered by maintenance therapy, such as, e.g., once a week for a period of 6 months or more.

For oral administration, the pharmaceutical composition of the invention can take the form of, for example, tablets or capsules prepared by conventional means with pharmaceutical acceptable excipients such as binding agents (e.g., pregelatinised maize starch, polyvinylpyrrolidone, hydroxypropyl methylcellulose), fillers (e.g., lactose, microcrystalline cellulose, calcium hydrogen phosphate), lubricants (e.g., magnesium stearate, talc, silica), disintegrants (e.g., potato starch, sodium starch glycolate), or wetting agents (e.g., sodium lauryl sulphate). Liquid preparations for oral administration can be in the form of, for example, solutions, syrups, or suspensions, or can be presented as a dry product for constitution with water or other suitable vehicle before use. Such liquid preparation can be prepared by conventional means with pharmaceutically acceptable additives such as suspending agents (e.g., sorbitol, syrup, cellulose derivatives, hydrogenated edible fats), emulsifying agents (e.g., lecithin, acacia), nonaqueous vehicles (e.g., almond oil, oily esters, ethyl alcohol, fractionated vegetable oils), preservatives (e.g., methyl or propyl-p-hydroxycarbonates, sorbic acids). The preparations can also contain buffer salts, flavouring, coloring and sweetening agents as deemed appropriate. Preparations for oral administration can be suitably formulated to give controlled release of the pharmaceutical composition of the invention.

The pharmaceutical composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

For administration by inhalation, the pharmaceutical composition of the invention is conveniently delivered in the form of an aerosol spray presentation from a pressurised pack or a nebulizer, with the use of a suitable propellant (e.g., dichlorodifluoromethane, trichlorofluoromethane, dichlorotetrafluoroethane, carbon dioxide, nitrogen, or other suitable gas). In the case of a pressurised aerosol, the dosage unit can be determined by providing a valve to deliver a metered amount. Capsules and cartridges of, for example, gelatine, for use in an inhaler or insufflator can be formulated containing a powder mix of the pharmaceutical composition of the invention and a suitable powder base such as lactose or starch.

The pharmaceutical composition of the invention can be formulated for parenteral administration by injection, for example, by bolus injection or continuous infusion. Formulations for injection can be presented in units dosage form (e.g., in phial, in multi-dose container), and with an added preservative. The pharmaceutical composition of the invention can take such forms as suspensions, solutions or emulsions in oily or aqueous vehicles, and can contain formulatory agents such as suspending, stabilizing, or dispersing agents. Alternatively, the agent can be in powder form for constitution with a suitable vehicle (e.g., sterile pyrogen-free water) before use. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilised powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Therapeutic/pharmaceutical compositions can be administered with medical devices known in the art. For example, in a preferred embodiment, a therapeutic/pharmaceutical composition of the invention can be administered with a needleless hypodermic injection device, such as the devices disclosed in U.S. Pat. Nos. 5,399,163; 5,383,851; 5,312,335; 5,064,413; 4,941,880; 4,790,824; or U.S. Pat. No. 4,596,556. Examples of well-known implants and modules useful in the present invention include those described in: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system.

Many other such implants, delivery systems, and modules are known to those skilled in the art. In certain embodiments, the compounds of the invention can be formulated to ensure proper distribution in vivo. For example, the blood-brain barrier (BBB) excludes many highly hydrophilic compounds. To ensure that the compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, and thus enhance targeted drug delivery (see, e.g., V. V. Ranade (1989) J. Clin. Pharmacol. 29: 685). Exemplary targeting moieties include folate or biotin (see, e.g., U.S. Pat. No. 5,416,016 to Low et al.); mannosides (Umezawa et al., (1988) Biochem. Biophys. Res. Commun. 153: 1038); antibodies (P. G. Bloeman et al. (1995) FEBS Lett. 357: 140; M. Owais et al. (1995) Antimicrob. Agents Chemother. 39: 180); and surfactant protein A receptor (Briscoe et al. (1995) Am. J. Physiol. 1233: 134).

In one embodiment of the invention, the compounds of the invention are formulated in liposomes. In a more preferred embodiment, the liposomes include a targeting moiety. In a most preferred embodiment, the compounds in the liposomes are delivered by bolus injection to a site proximal to the desired area. Such liposome-based composition should be fluid to the extent that easy syringability exists, should be stable under the conditions of manufacture and storage and should be preserved against the contaminating action of microorganisms such as bacteria and fungi.

### EXAMPLES OF THE INVENTION

### 1. Example 1

### 1.1 Material & Methods

### Cell Culture

Cell lines: Human fibrosarcoma cell line HT-1080 and immortalized Mouse Embryonic Fibroblasts (MEF). MEFs were a gift from Daniel Krappmann, HT-1080 were purchased from ATCC and both cell lines were grown in Dulbecco's Modified Eagle's medium (Thermo Fisher Scientific, 41966-029) supplemented with 10% fetal bovine serum (FBS, Thermo Fisher Scientific), 1% Penicillin-Streptomycin (Thermo Fisher Scientific) and 1% nonessential amino acids (MEM NEAA, Thermo Fisher Scientific). Cells were grown at 37° C. and 5% CO₂.

### Compounds

For ferroptosis assays, (1S,3R)-RSL3 (RSL3, Sigma), Imidazole Ketone Erastin (IKE, Cayman Chemical), FIN56 (Cayman Chemical), FINO₂ (Cayman Chemical), Seratrodast (Sera, Cayman Chemical) and Ferrostatin-1 (Fer-1, Sigma) were purchased.

For the apoptosis and necroptosis assay, Staurosporine (Stauro, TargetMol) and Z-VAD-FMK (zVAD, TargetMol), LCL161 (MedChemExpress), Tumor Necrosis Factor alpha (TNFα, biomol) and Necrostatin-1 (Nec-1, BioVision) were purchased.

Following TXA2 receptor antagonists were purchased: Terutroban (Sigma), Vapiprost (Santa Cruz Biotechnology), Ramatroban (BAYu 3405, Santa Cruz Biotechnology) and 15(R)-Pinane Thromboxane A₂ (PTA2, Cayman Chemical).

### Cell Viability Assays

HT-1080 were seeded into 384-well plates (CulturPlates, PerkinElmer) with a density of 750 cells per well. After 24 h of growth, cells were treated with 10 or 12-point serial dilutions of compounds in indicated concentrations for 18 h. Viability was detected by using CellTiter-Glo® 2.0 Regent (Promega) according to the manufacturer's instruction and luminescence was read out in an EnVison 2104 Multilabel plate reader (PerkinElmer).

For detecting apoptosis, HT-1080 were treated with Staurosporine before addition of Seratrodast or zVAD-FMK. Caspase 3/7 activity was measured 18 hours later via Caspase-Glo® 3/7 Assay Reagent (Promega) according to the manufacturer's instruction. Cells were incubated for 45 min at room temperature and luminescence was measured.

For detecting necroptosis, MEF were treated with TNFα, zVAD-FMK and LCL161 before addition of Seratrodast or Necrostatin-1. Cell viability was measured after 18 hours by adding CellTiter-Glo® 2.0 Reagent (Promega) according to the manufacturer's instruction and luminescence was read out.

### Spheroid Formation and Imaging

2,000 HT-1080 cells were seeded per well into a 96-well Round Bottom Ultra Low Attachment Microplate (Corning costar 7007). Over a period of 48 h spheroids were formed and subsequently treated with RSL3, and Ferrostatin-1 or Seratrodast. After another 48 hours, spheroids were stained using Hoechst 33342 (Sigma) in a 1:10,000 dilution and incubated for 1 h. Images were acquired using an Operetta high-content imaging system (PerkinElmer) and analyses were conducted with the Columbus software (PerkinElmer). For analysis, spheroids were detected as “Image region” and morphology properties were calculated (roundness).

### Cell-Free BODIPY Assay

Ferrostatin-1 and Seratrodast were diluted in 150 μl PBS to achieve a concentration of 25 μM. As a control, the same amount of DMSO was diluted in 150 μl PBS. In separate tubes, BODIPY 581/591 C11 (Thermo Fisher Scientific) was diluted to 1.875 μM in 150 μl PBS and 2,2′-Azobis(2-methylpropionamidine) dihydrochloride (AAPH, Sigma) was diluted to 7.5 mM in 150 μl PBS. For the non-oxidized control, one sample was prepared as DMSO only without AAPH. All three solutions were mixed equally and incubated for 30 min at room temperature in the dark. 100 μl of each condition were transferred into a black 96-well plate (Greiner Bio-One) and fluorescence was measured at 495 nm/520 nm in an EnVision 2104 Multilabel plate reader (PerkinElmer).

### TBARS Assay

2 million HT-1080 cells were seeded into 150 mm dishes and grown for 48 hours. Ferroptosis was induced with RSL3 for 2.5 hours and cells were co-treated with Ferrostatin-1 or Seratrodast. Cells were detached by 0.05% Trypsin-EDTA (Thermo Fisher Scientific) and cell number was adjusted to the sample with the lowest cell count. For measuring MDA levels, TBARS (TCA Method) Assay Kit (Cayman Chemical, 700870) was used according to manufacturer's instruction. Fluorescence was detected at 530 nm/550 nm in an EnVision 2104 Multilabel plate reader (PerkinElmer).

### Flow Cytometry

HT1080 cells were grown in 6-well plates (200,000 cells per well) and incubated for 24 h. For staining with BODIPY 581/591 C11 (Thermo Fisher Scientific), cells were treated with RSL3, and Ferrostatin-1 or Seratrodast for 2 h, then BODIPY was added into the wells to a final concentration of 2 μM. After 30 min incubation, cells were detached using Trypsin and washed two times with PBS. Between the wash steps, cells were centrifuged at 500×g for 5 minutes. After the final washing step, cell pellets were resuspended in 300 μl PBS and 10,000 events per condition were analyzed in the BL-1 channel of an Attune acoustic flow cytometer (Applied Biosystems). For detection of 4-Hydroxynonenal (4-HNE), HT-1080 were induced with RSL3, and co-treated with Ferrostatin-1 or Seratrodast for 2 h before they were detached using Trypsin. Cell pellets were incubated in 10% normal goat serum (Thermo Fisher Scientific) for 30 min on ice. Subsequently, cells were incubated with anti-4-HNE antibody (1:50 dilution in 1% BSA in PBS, ab46545, Abcam) for 1 h on ice. After three washing steps with PBS, cells were resuspended in anti-rabbit Alexa 488 antibody (1:200 dilution in 1% BSA in PBS, A32731, Thermo Fisher Scientific) and incubated for 30 min on ice. For flow cytometry, cells were collected in 300 μl PBS and 10,000 events per condition were analyzed in an Attune acoustic flow cytometer (Applied Biosystems), using the BL-1 channel. FlowJo™ v10.8.1 (BD Life Sciences) was used for creating histograms and analyzing fluorescence intensity.

### Statistics

Statistical analysis was performed using GraphPad Prism version 9.4. Dose-response curves were performed in 2 biological replicates, each experiment having two technical replicates per condition. Apoptosis and necroptosis assays were performed in technical triplicates. The spheroid experiment was performed with n=8 spheroids for every condition. The TXA2 receptor antagonist assay was performed in technical triplicates. The TBARS assay, cell-free BODIPY Assay, 4-HNE immunostaining and BODIPY staining were performed in 3 independent biological experiments. For FACS experiments, representative histograms are shown and all histograms quantified for bar graph representation (n=3). Statistical test is indicated in the Figure legend.

### 1.2 Results and Discussion

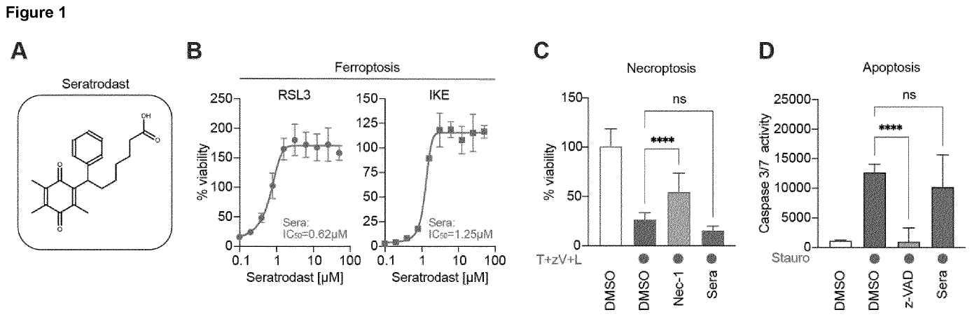

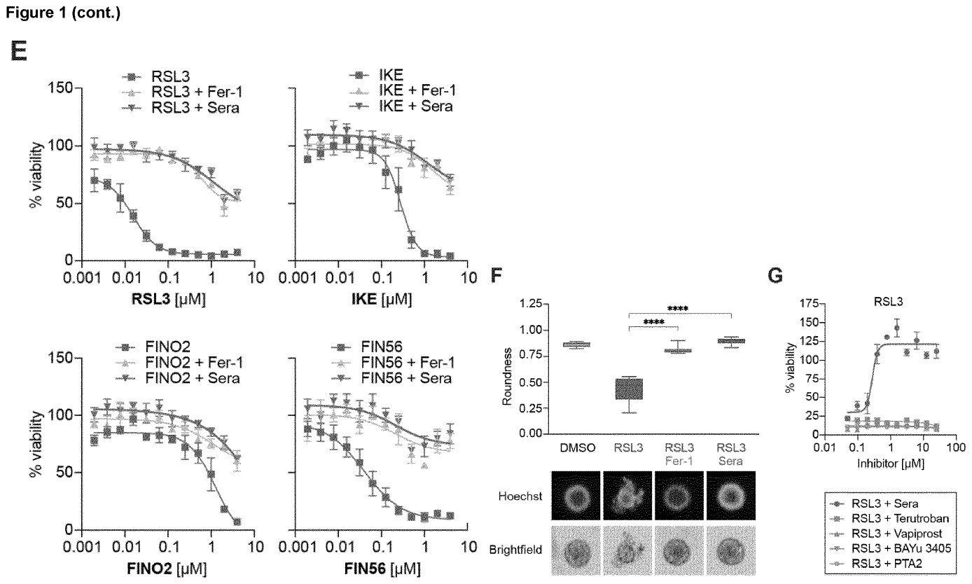

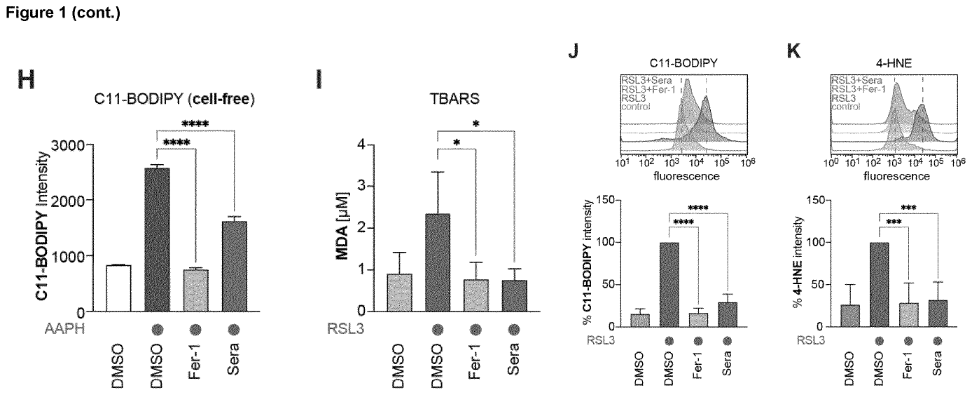

It has been rationally looked for previously clinically-applied small molecules with potential radical-trapping capacity to be used in our drug repurposing approach to block ferroptosis. Seratrodast (FIG. 1A) has been studied, a thromboxane A2 receptor (TXA2) antagonist (13), which contains a quinone moiety and investigated whether it could inhibit ferroptosis in our model cell line HT-1080. The effects of Seratrodast on different cell death modalities have been studied. Intriguingly, Seratrodast suppressed ferroptosis (FIG. 1B), but not necroptosis (FIG. 1C) and apoptosis (FIG. 1D); thus, demonstrating good selectively towards ferroptosis over the other two tested regulated cell death pathways. Next, various ferroptosis inducers (FINs) have been used to understand if Seratrodast universally counteracts ferroptosis. To this end, ferroptosis with IKE (Class I FIN), RSL3 (Class II FIN), FIN56, and FINO₂ has been induced. Seratrodast was able to suppress ferroptosis induced by all four FINs (FIG. 1E), and importantly was as potent as ferrostatin-1 (9), a reference ferroptosis inhibitor. It has been further analyzed if Seratrodast would compete ferroptotic cell death in a 3D spheroid system that encompasses a more physiological setting rather than 2D mono-layer culture. Treatment of HT-1080-derived spheroids with RSL3 led to dissociation of the 3D organization, which was quantified by exhibiting less roundness (FIG. 1F). Ferrostatin-1 as well as Seratrodast fully inhibited ferroptosis-based spheroid destruction (FIG. 1F). To understand the mechanism behind the ferroptosis-inhibitory potential of Seratrodast, additional TXA2 receptor antagonists lacking a quinone moiety (in contrast to Seratrodast) to uncouple radical-trapping activity from TXA2 receptor inhibition have been tested. Interestingly, all our TXA2 receptor inhibitors without a quinone moiety were unable to compete ferroptotic cells death, thus arguing for the radical-trapping activity of Seratrodast to inhibit ferroptosis (FIG. 1G). To validate this finding, a cell-free C11-BODIPY experiment has been performed by using free-radical-producing 2,2′-azobis(2-methyl-propanimidamide) dihydrochloride (AAPH) together with the C11-BODIPY sensor. Seratrodast as well as Fer-1 both significantly reduced oxidative fluorescence induced by AAPH, again demonstrating radical-trapping capacity of Seratrodast (FIG. 1H). Consequently, Seratrodast eliminated levels of malondialdehyde (MDA)—a product of lipid peroxidation induced upon RSL3 treatment (FIG. 1I). Similarly, levels of RSL3-mediated peroxidation of the C11-BODIPY sensor were largely reduced upon Seratrodast treatment (FIG. 1J). Finally, 4-hydroxynonenal (4-HNE) levels—generated upon RSL3 treatment—were reduced by Seratrodast (FIG. 1K). Notably, all ferroptosis-inhibitory effects of Seratrodast on cell viability as well as lipid peroxidation were similar to the reference molecule ferrostatin-1.

### 2. Example 2

Based on the assays of example 1, further quinones have been tested, as listed in table 2.

| | | | IC50 | | cellular | | |
|---|---|---|---|---|---|---|---|
| | | | (Ferroptosis | | efficacy/toxicity | Apoptosis | Necroptosis |
| Name | Code | Structure | inhibition) | cytotoxic IC50 | window | inhibition | inhibition |
| Seratrodast | iQFerr01 | 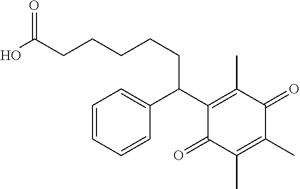 [SMILES: CC1=C(C)C(=O)C(C(CCCCCC(=O)O)c2ccccc2)=C(C)C1=O] | 290 nM | 57910 nM | 200-fold | no | no |
| Shikonin | iQFerr03 | 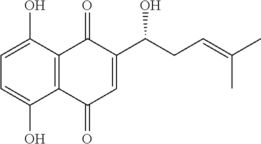 [SMILES: CC(C)=CC[C@@H](O)C1=CC(=O)c2c(O)ccc(O)c2C1=O] | >1000 nM | 269 nM* | no efficacy | | |
| Alkannin | iQFerr04 | 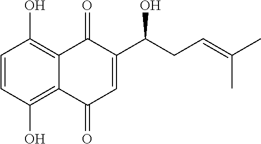 [SMILES: CC(C)=CC[C@H](O)C1=CC(=O)c2c(O)ccc(O)c2C1=O] | >1000 nM | 573 nM* | no efficacy | | |
| Deoxyshikonin | iQFerr05 | 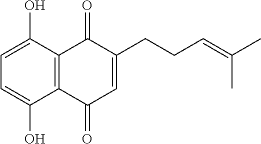 [SMILES: CC(C)=CCCC1=CC(=O)c2c(O)ccc(O)c2C1=O] | 287 nM | 1625 nM* | 6-fold | | |
| Lapachol | iQFerr06 | 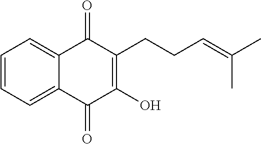 [SMILES: CC(C)=CCCC1=C(O)C(=O)c2ccccc2C1=O] | 261 nM | 6581 nM* | 25-fold | | |
| Coenzyme Q1 | iQFerr07 | 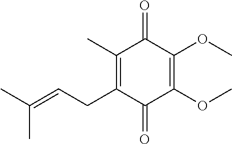 [SMILES: COC1=C(OC)C(=O)C(CC=C(C)C)=C(C)C1=O] | 30 nM | 10000 nM | 333-fold | | |
| Anthraquinone-2- carboxyclic acid | iQFerr08 | 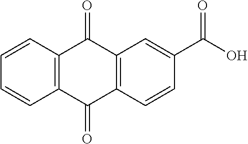 [SMILES: O=C(O)c1ccc2c(c1)C(=O)c1ccccc1C2=O] | 13000 nM | >100 μM | low efficacy | | |
| NQ301 | iQFerr02 | 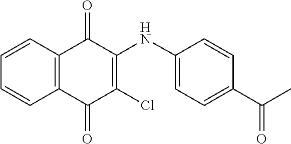 [SMILES: CC(=O)c1ccc(NC2=C(Cl)C(=O)c3ccccc3C2=O)cc1] | 27 nM | 759 nM* | 28-fold | >800 nM | no |
| 2-(3-acetylanilino)-3- chloro-1,4- naphtoquinone | iQFerr09 | 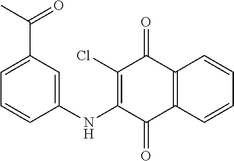 [SMILES: CC(=O)c1cccc(NC2=C(Cl)C(=O)c3ccccc3C2=O)c1] | 63 nM | 810 nM | 13-fold | | |
| 2-chloro-3-(4- ethylanilino)-1,4- naphtoquinone | iQFerr10 | 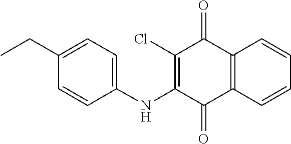 [SMILES: CCc1ccc(NC2=C(Cl)C(=O)c3ccccc3C2=O)cc1] | 8 nM | 844 nM | 105-fold | | |
| 2-chloro-3-(4-hydroxy- phenylamino)-1,4- naphtoquinone | iQFerr11 | 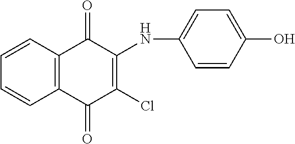 [SMILES: O=C1C(Cl)=C(Nc2ccc(O)cc2)C(=O)c2ccccc21] | 6 nM | 293 nM | 48-fold | | |
| 2-chloro-3-(4- methoxyanilino)-1,4- naphtoquinone | iQFerr12 | 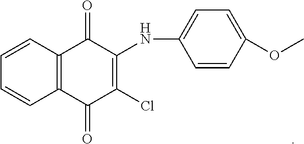 [SMILES: COc1ccc(NC2=C(Cl)C(=O)c3ccccc3C2=O)cc1] | 6 nM | 3516 nM | 586-fold | | |
| 2-chloro-3-(4- methylanilino)-1,4- naphtoquinone | iQFerr13 | 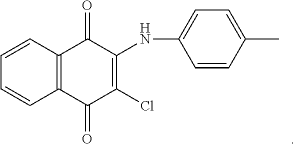 [SMILES: Cc1ccc(NC2=C(Cl)C(=O)c3ccccc3C2=O)cc1] | 3 nM | 380 nM | 126-fold | | |
| 2-chloro-3-(2,4- dimethylanilino)-1,4- naphtoquinone | iQFerr14 | 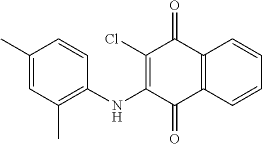 [SMILES: Cc1ccc(NC2=C(Cl)C(=O)c3ccccc3C2=O)c(C)c1] | 0.034 nM | 1003 nM * | 5000-fold | > 800 nM | |
| 4-[(3-chloro-1,4-dioxo- 1,4-dihydronaphthalen-2- yl)amino]butanoic acid | iQFerr15 | 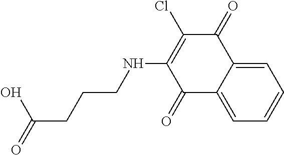 [SMILES: O=C(O)CCCNC1=C(Cl)C(=O)c2ccccc2C1=O] | 1356 nM | 215000 nM | 159-fold | | |
| 2-anilino-3-chloro-1,4- naphtoquinone | iQFerr16 | 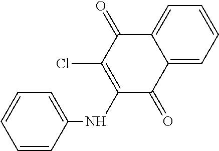 [SMILES: O=C1C(Cl)=C(Nc2ccccc2)C(=O)c2ccccc21] | 4.5 nM | 279 nM | 62-fold | | |
| 2-chloro-3-(4- fluoroanilino)-1,4- naphtoquinone | iQFerr17 | 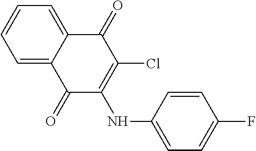 [SMILES: O=C1C(Cl)=C(Nc2ccc(F)cc2)C(=O)c2ccccc21] | 6.1 nM | 397 nM | 65-fold | | |
| 4-[(3-chloro-1,4-dioxo- 1,4-dihydronaphthalen-2- yl)amino]benzoic acid | iQFerr18 | 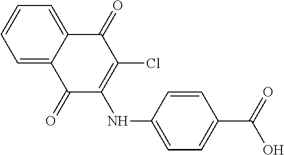 [SMILES: O=C(O)c1ccc(NC2=C(Cl)C(=O)c3ccccc3C2=O)cc1] | 94.12 nM | 2546 nM | 27-fold | | |
| 2-[4-(dimethylamino)- anilino]-1,4- naphtoquinone | iQFerr19 | 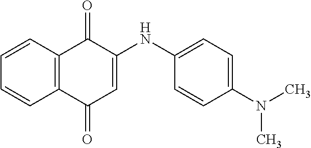 [SMILES: CN(C)c1ccc(NC2=CC(=O)c3ccccc3C2=O)cc1] | 32.17 nM | 5849 nM | 182-fold | | |
| N-[4-(1,4-dioxo-1,4- dihydronaphthalen-2-yl- amino)phenyl]acetamide | iQFerr20 | 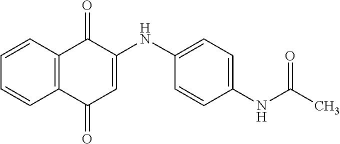 [SMILES: CC(=O)Nc1ccc(NC2=CC(=O)c3ccccc3C2=O)cc1] | 8.12 nM | 10180 nM* | 1254-fold | no | no |
| 2-(4-ethoxyanilino)-1,4- naphtoquinone | iQFerr21 | 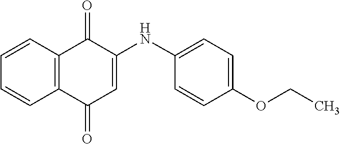 [SMILES: CCOc1ccc(NC2=CC(=O)c3ccccc3C2=O)cc1] | 43.03 nM | 8567 nM | 199-fold | | |
| 2-(4-methylanilino)-1,4- naphtoquinone | iQFerr22 | 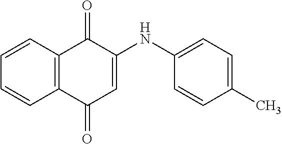 [SMILES: Cc1ccc(NC2=CC(=O)c3ccccc3C2=O)cc1] | 15.55 nM | 5499 nM | 354-fold | | |
| 2-[(5-hydroxy-1,4-dioxo- 1,4-dihydronaphthalen-2- yl-amino]benzoic acid | iQFerr23 | 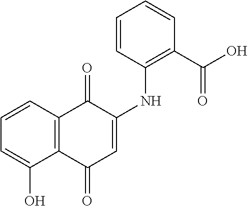 [SMILES: O=C(O)c1ccccc1NC1=CC(=O)c2c(O)cccc2C1=O] | 3479 nM | 16400 nM | 5-fold | | |
| 1,3-diphenyl-1H-benzo-f- indazole-4,9-dione | iQFerr24 | 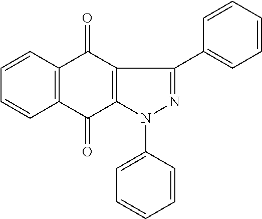 [SMILES: O=C1c2ccccc2C(=O)c2c1c(-c1ccccc1)nn2-c1ccccc1] | >100 000 nM | <0.001 nM | no efficacy | | |
| 2-(2,4-dimethylanilino)- benzo-1,4-quinone | iQFerr25 | 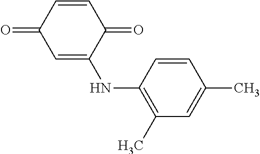 [SMILES: Cc1ccc(NC2=CC(=O)C=CC2=O)c(C)c1] | 175.7 nM | 759.3 nM | 5-fold | | |
| 2,2-(3,6-dioxo-1,4- cyclohexadiene-1,4-diyl- diimino)dibenzoic acid | QFerr26 | 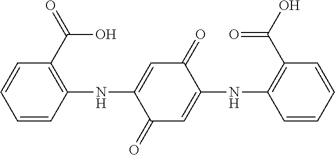 [SMILES: O=C1C=C(Nc2ccccc2C(=O)O)C(=O)C=C1Nc1ccccc1C(=O)O] | >100 000 nM | 35430 nM | no efficacy | | |
| 2-(4-ethoxyanilino)- benzo-1,4-quinone | iQFerr27 | 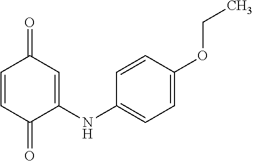 [SMILES: CCOc1ccc(NC2=CC(=O)C=CC2=O)cc1] | 493.1 nM | 2157 nM | 4-fold | | |
| 2-(4-fluorobenzyl)-1,4- naphtoquinone | iQFerr28 | 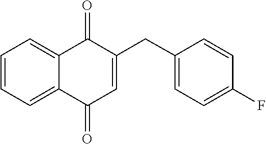 [SMILES: O=C1C=C(Cc2ccc(F)cc2)C(=O)c2ccccc21] | 32.42 nM | 1451 nM | 45-fold | | |
| 2-diphenylmethyl-1,4- naphtoquinone | iQFerr29 | 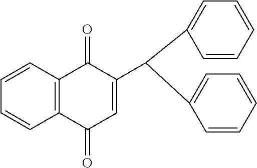 [SMILES: O=C1C=C(C(c2ccccc2)c2ccccc2)C(=O)c2ccccc21] | 3.909 nM | 4993 nM | 1277-fold | >10000 nM | no |
| 2-(4-methylbenzyl)-1,4- naphtoquinone | iQFerr30 | 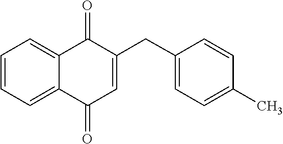 [SMILES: Cc1ccc(CC2=CC(=O)c3ccccc3C2=O)cc1] | 38.37 nM | 5056 nM | 132-fold | | |
| 2-(4-methoxybenzyl)-1,4- naphtoquinone | iQFerr31 | 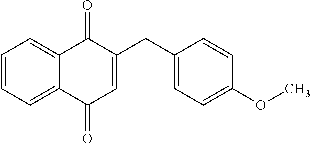 [SMILES: COc1ccc(CC2=CC(=O)c3ccccc3C2=O)cc1] | 1056 nM | 8703 nM | 8-fold | | |
| 2-hydroxy-3-[(4- methylphenyl)phenyl- methyl]-1,4- naphtoquinone | iQFerr32 | 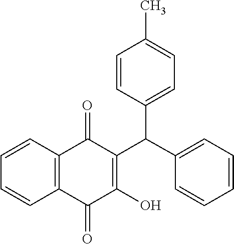 [SMILES: Cc1ccc(C(C2=C(O)C(=O)c3ccccc3C2=O)c2ccccc2)cc1] | 39800 nM | 34210 nM | no efficacy | | |
| 2-[(4-dimethylamino- phenyl)phenylmethyl]-3- hydroxy-1,4- naphtoquinone | iQFerr33 | 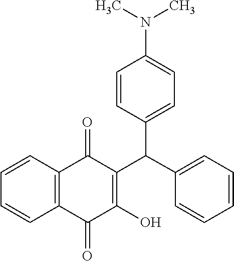 [SMILES: CN(C)c1ccc(C(C2=C(O)C(=O)c3ccccc3C2=O)c2ccccc2)cc1] | 7577 nM | 71850 nM | 9-fold | | |
| 2-[(4-fluorophenyl)- phenylmethyl]-3-hydroxy 1,4-naphtoquinone | iQFerr34 | 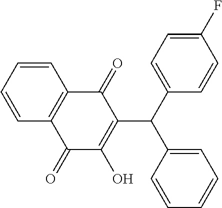 [SMILES: O=C1C(O)=C(C(c2ccccc2)c2ccc(F)cc2)C(=O)c2ccccc21] | 71390 nM | 35040 nM | no efficacy | | |
| 2-chloro-3-[(2,4- dimethylphenyl)amino]- 1,4-dihydronaphthalene- 1,4-dione | iQFerr35 | 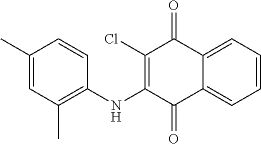 [SMILES: Cc1ccc(NC2=C(Cl)C(=O)c3ccccc3C2=O)c(C)c1] | 0.6918 nM | 5570 nM | 8051-fold | | |
| N-{4-[(3-chloro-1,4-dioxo- 1,4-dihydronaphthalen-2 yl)amino]phenyl} acetamide | iQFerr36 | 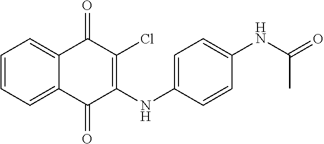 [SMILES: CC(=O)Nc1ccc(NC2=C(Cl)C(=O)c3ccccc3C2=O)cc1] | 7.355 nM | 564 nM | 77-fold | | |
| 2-[(2,4- dimethylphenyl)amino]- 1,4-dihydronaphthalene- 1,4-dione | iQFerr37 | 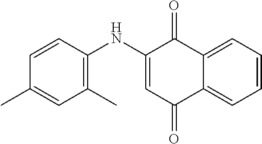 [SMILES: Cc1ccc(NC2=CC(=O)c3ccccc3C2=O)c(C)c1] | 15.43 nM | 48790 nM | 3162-fold | | |
| 6-[(2,4- dimethylphenyl)(1,4- dioxo-1,4- dihydronaphthalen-2- yl)amino]hexanoic acid | iQFerr38 | 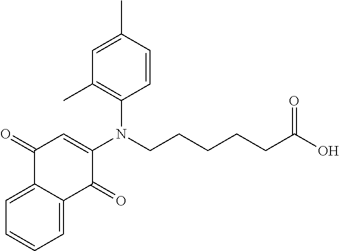 [SMILES: Cc1ccc(N(CCCCCC(=O)O)C2=CC(=O)c3ccccc3C2=O)c(C)c1] | 214.8 nM | 49800 nM* | 232-fold | no | no |
| N-{4-[(3-methyl-1,4- dioxo-1,4- dihydronaphthalen-2- yl)amino]phenyl} acetamide | iQFerr39 | 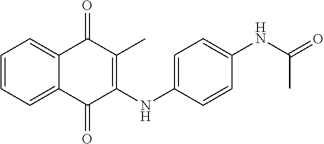 [SMILES: CC(=O)Nc1ccc(NC2=C(C)C(=O)c3ccccc3C2=O)cc1] | 17 nM | 43540 nM* | 2561-fold | no | no |
| 4-[(2,4- dimethylphenyl)(1,4- dioxo-1,4- dihydronaphthalen-2- yl)amino]butanamide | iQFerr40 | 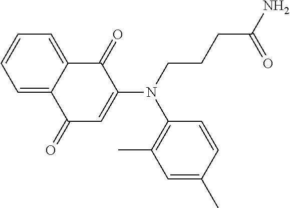 [SMILES: Cc1ccc(N(CCCC(N)=O)C2=CC(=O)c3ccccc3C2=O)c(C)c1] | 188.8 nM | 229.7 nM | 1,2-fold | | |
| 2-[(2,4- dimethylphenyl)amino]- 3-methyl-1,4- dihydronaphthalene-1,4- dione | iQFerr41 | 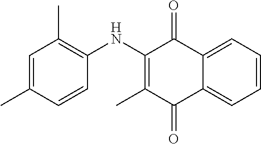 [SMILES: CC1=C(Nc2ccc(C)cc2C)C(=O)c2ccccc2C1=O] | 2.91 nM | 13390 nM* | 4601-fold | no | no |
| 4-[(2,4- dimethylphenyl)(1,4- dioxo-1,4- dihydronaphthalen-2- yl)amino]butanoic acid | iQFerr42 | 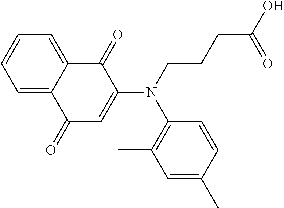 [SMILES: Cc1ccc(N(CCCC(=O)O)C2=CC(=O)c3ccccc3C2=O)c(C)c1] | 463 nM | 219 nM | 0,47-fold | | |
| 6-[(2,4- dimethylphenyl)(1,4- dioxo-1,4- dihydronaphthalen-2- yl)amino]hexanamide | iQFerr43 | 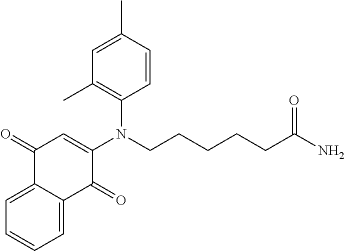 [SMILES: Cc1ccc(N(CCCCCC(N)=O)C2=CC(=O)c3ccccc3C2=O)c(C)c1] | 115.6 nM | 75450 nM* | 653-fold | no | no |
| N-(4-{[1,4-dioxo-3- (trifluoromethyl)-1,4- dihydronaphthalen-2- yl]amino}phenyl) acetamide | iQFerr44 | 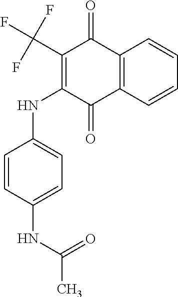 [SMILES: CC(=O)Nc1ccc(NC2=C(C(F)(F)F)C(=O)c3ccccc3C2=O)cc1] | 30.92 nM | 58790 nM* | 1901-fold | >6000 nM | no |
| 2-methyl-3-[(4- methylphenyl)amino]- 1,4-dihydronaphthalene- 1,4-dione | iQFerr45 | 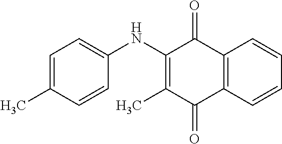 [SMILES: CC1=C(Nc2ccc(C)cc2)C(=O)c2ccccc2C1=O] | 1.56 nM | 39990 nM | 25634-fold | | |
| 2-[(3,5- dimethylphenyl)amino]- 3-methyl-1,4- dihydronaphthalene-1,4- dione | iQFerr46 | 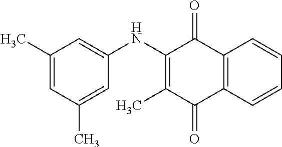 [SMILES: CC1=C(Nc2cc(C)cc(C)c2)C(=O)c2ccccc2C1=O] | 2.11 nM | 77560 nM* | 36758-fold | no | no |
| 2-[(2,4- dimethylphenyl)amino]- 3-(trifluoromethyl)-1,4- dihydronaphthalene-1,4- dione | iQFerr47 | 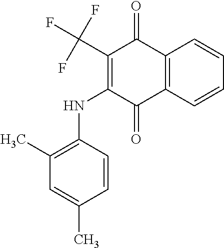 [SMILES: Cc1ccc(NC2=C(C(F)(F)F)C(=O)c3ccccc3C2=O)c(C)c1] | 10.08 nM | 14700 nM* | 1458-fold | no | no |
| 6-[(1,4-dioxo-1,4- dihydronaphthalen-2- yl)(4- acetamidophenyl)amino] hexanoic acid | iQFerr48 | 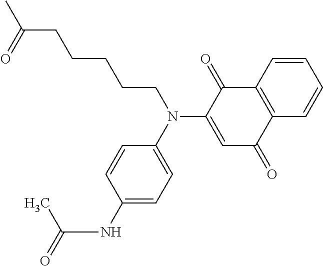 [SMILES: CC(=O)CCCCCN(C1=CC(=O)c2ccccc2C1=O)c1ccc(NC(C)=O)cc1] | 619 nM | 50040 nM* | 80-fold | | |
| 6-[(1,4-dioxo-1,4- dihydronaphthalen-2- yl)(4- acetamidophenyl)amino] hexanamide | iQFerr49 | 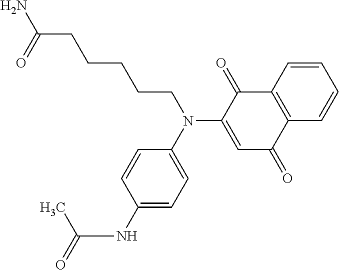 [SMILES: CC(=O)Nc1ccc(N(CCCCCC(N)=O)C2=CC(=O)c3ccccc3C2=O)cc1] | 22.84 nM | 111800 nM* | 4895-fold | | |

*Signal quenching at higher concentration. Thus, toxicity might be less than indicated

### REFERENCES

1. S. J. Dixon et al., Ferroptosis: an iron-dependent form of nonapoptotic cell death. Cell 149, 1060-1072 (2012).
2. B. R. Stockwell, Ferroptosis turns 10: Emerging mechanisms, physiological functions, and therapeutic applications. Cell 185, 2401-2421 (2022).
3. K. Bersuker et al., The CoQ oxidoreductase FSP1 acts parallel to GPX4 to inhibit ferroptosis. Nature 575, 688-692 (2019).
4. S. Doll et al., FSP1 is a glutathione-independent ferroptosis suppressor. Nature 575, 693-698 (2019).
5. V. A. N. Kraft et al., GTP Cyclohydrolase 1/Tetrahydrobiopterin Counteract Ferroptosis through Lipid Remodeling. ACS central science 6, 41-53 (2020).
6. M. Soula et al., Metabolic determinants of cancer cell sensitivity to canonical ferroptosis inducers. Nat Chem Biol 16, 1351-1360 (2020).
7. C. Mao et al., DHODH-mediated ferroptosis defence is a targetable vulnerability in cancer. Nature 593, 586-590 (2021).
8. E. Mishima et al., A non-canonical vitamin K cycle is a potent ferroptosis suppressor. Nature 10.1038/s41586-022-05022-3 (2022).
9. K. Hadian, B. R. Stockwell, SnapShot: Ferroptosis. Cell 181, 1188-1188 e1181 (2020).
10. M. Conrad, S. M. Lorenz, B. Proneth, Targeting Ferroptosis: New Hope for As-Yet-Incurable Diseases. Trends in molecular medicine 27, 113-122 (2020).
11. K. Hadian, B. R. Stockwell, A roadmap to creating ferroptosis-based medicines. Nat Chem Biol 17, 1113-1116 (2021).
12. S. Pushpakom et al., Drug repurposing: progress, challenges and recommendations. Nat Rev Drug Discov 18, 41-58 (2019).
13. S. Terao, M. Shiraishi, T. Matsumoto, Y. Ashida, [Thromboxane A2 antagonist-discovery of seratrodast]. Yakugaku Zasshi 119, 377-390 (1999)

1. A quinone for use in the inhibition of ferroptosis.

2. The quinone of claim 1 for use in the treatment of a disease which can be treated or prevented by inhibition of ferroptosis.

3. The quinone for use of claim 2, wherein the disease is selected from the group consisting of Amyotrophic Lateral Sclerosis (ALS), Alzheimer's Disease (AD), Parkinson's Disease (PD), Huntington's Disease (HD), COPD, stroke, traumatic brain injury, kidney degeneration, acute kidney failure, heart damage, cardiomyopathy, acute liver failure, lung damage, and retinal degeneration.

4. The quinone for use of claims 1 to 3, wherein the quinone is selected from a quinone as specified in one of the general formula (I), or (II) 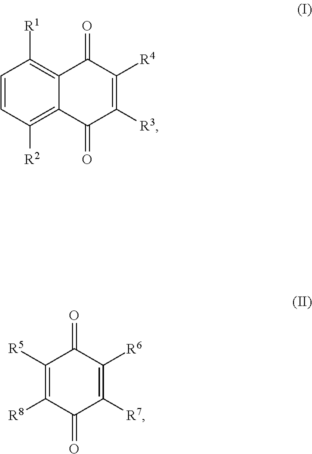
R¹ and R² are independently selected from the group consisting of H and —OH;
R³ is selected from the group consisting of H, —(C₁-C₃₀)alkyl, —(C₂-C₁₂)alkenyl, —OH, chlorine, —O(C₁-C₁₀)alkyl, trifluoromethyl;
R⁴ is independently selected from the group consisting of —(C₁-C₃₀)alkyl, —(C₂-C₁₂)alkenyl, —NH(C₁-C₁₀)alkyl and —NH(C₆-C₁₂)aryl, 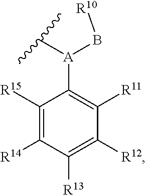
wherein in (C₁-C₃₀)alkyl, —(C₂-C₁₂)alkenyl, —NH(C₁-C₁₀)alkyl and —NH(C₆-C₁₂)aryl one or more hydrogen atoms are optionally independently substituted by a substituent selected from the group consisting of —OH, —O(C₁-C₁₀)alkyl, (C₁-C₁₀)alkyl, —C(O)(C₁-C₁₀)alkyl, —COO(C₁-C₁₀)alkyl, —CONH₂, —COOH, —(C₆-C₁₂)aryl;
R⁵ is selected from the group consisting of H, —(C₁-C₃₀)alkyl, —(C₂-C₁₂)alkenyl, —OH, and chlorine, —O(C₁-C₁₀)alkyl, trifluoromethyl;
R⁶ is independently selected from the group consisting of —(C₁-C₃₀)alkyl, —(C₂-C₁₂)alkenyl, —NH(C₁-C₁₀)alkyl and —NH(C₆-C₁₂)aryl, 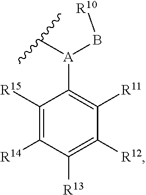
wherein in (C₁-C₃₀)alkyl, —(C₂-C₁₂)alkenyl, —NH(C₁-C₁₀)alkyl and —NH(C₆-C₁₂)aryl one or more hydrogen atoms are optionally independently substituted by a substituent selected from the group consisting of —OH, —O(C₁-C₁₀)alkyl, (C₁-C₁₀)alkyl, —C(O)(C₁-C₁₀)alkyl, —COO(C₁-C₁₀)alkyl, —CONH₂, —COOH, —(C₆-C₁₂)aryl.
R⁷ is selected from the group consisting of H, —(C₁-C₃₀)alkyl, —(C₂-C₁₂)alkenyl, —OH, chlorine, —O(C₁-C₁₀)alkyl, trifluoromethyl;
R⁸ is selected from the group consisting of H, —(C₁-C₃₀)alkyl, —(C₂-C₁₂)alkenyl, —OH, chlorine, —O(C₁-C₁₀)alkyl, trifluoromethyl;
A is N or CR¹⁶;
B is a bond, (C₁-C₈)alkylene, or (C₂-C₈)alkenylene;
R¹⁰ is selected from the group consisting of H, —COOH, —CONH₂, —COO(C₁-C₆)alkyl;
R¹¹, R¹², R¹³, R¹⁴, R¹⁵ are independently selected from the group consisting of H, (C₁-C₄) alkyl, —O(C₁-C₄)alkyl, —OH, halogen, —CF₃, —CO(C₁-C₄)alkyl, COOH, —COO(C₁-C₆)alkyl, —CONH₂, —NH₂, —NHCO(C₁-C₄)alkyl, —NH(C₁-C₄)alkyl, and —N((C₁-C₄)alkyl)₂; preferably H, —(C₁-C₄) alkyl, —O(C₁-C₄)alkyl, —OH, Cl, F, —CF₃, —CO(C₁-C₄)alkyl, and COOH; more preferably H, —(C₁-C₄) alkyl, —O(C₁-C₄)alkyl, —OH, Cl, —CO(C₁-C₄)alkyl, and COOH;
R¹⁶ is H, (C₁-C₄) alkyl, OH; preferably, H, CH₃ or OH;
or a pharmaceutically acceptable salt, solvate, enantiomer, diastereomer, racemate or hydrate thereof.

5. The quinone for use of claim 4, wherein the quinone is selected from
quinone (I), wherein
R³ is selected from the group consisting of H, —(C₁-C₅)alkyl, —O(C₁-C₅)alkyl, —OH, trifluoromethyl, and Cl,
preferably, R³ is selected from the group consisting of H, —CH₃, —OH, —OMe, trifluoromethyl and Cl; more preferably R³ is selected from group consisting of H, —CH₃, —OH, trifluoromethyl, and C and/or
R¹¹, R¹², R¹³, R¹⁴, R¹⁵ are independently selected from the group consisting of H, —(C₁-C₄) alkyl, —OCH₃, —OH, Cl, F, —CF₃, —CO(C₁-C₄)alkyl, COOH;
Preferably R¹¹, R¹², R¹³, R¹⁴, R¹⁵ are independently selected from the group consisting of H, —(C₁-C₄) alkyl, —OCH₃, —OH, Cl, F, —CF₃, —CO(C₁-C₄)alkyl, COOH; more preferably R¹¹, R¹², R¹³, R¹⁴, R¹⁵ are independently selected from the group consisting of H, —(C₁-C₄) alkyl, —O(C₁-C₄)alkyl, —OH, Cl, —CO(C₁-C₄)alkyl, and COOH or
quinone (II), wherein
R⁵ is selected from the group consisting of H, —(C₁-C₁₀)alkyl, trifluoromethyl, —O(C₁-C₁₀)alkyl;
preferably, R⁵ is selected from the group consisting of H, —CH₃, —OCH₃; more preferably, R⁵ is selected from the group consisting of —CH₃, —OCH₃ and/or
R⁷ is selected from the group consisting of H, —(C₁-C₅)alkyl, —O(C₁-C₅)alkyl, —OH, trifluoromethyl and Cl;
preferably, R⁷ is selected from the group consisting of H, —CH₃, —OH, —OMe and Cl;
more preferably R⁷ is selected from group consisting of H, —CH₃, —OH, and Cl and/or
R⁸ is selected from the group consisting of H, —(C₁-C₁₀)alkyl, trifluoromethyl, —O(C₁-C₁₀)alkyl;
preferably, R⁸ is selected from the group consisting of H, —CH₃, —OCH₃; more preferably, R⁸ is selected from the group consisting of —CH₃, —OCH₃; and/or
R¹¹, R¹², R¹³, R¹⁴, R¹⁵ are independently selected from the group consisting of H, —(C₁-C₄) alkyl, —OCH₃, —OH, Cl, F, —CF₃, —CO(C₁-C₄)alkyl, COOH;
Preferably R¹¹, R¹², R¹³, R¹⁴, R¹⁵ are independently selected from the group consisting of H, —(C₁-C₄) alkyl, —OCH₃, —OH, Cl, F, —CF₃, —CO(C₁-C₄)alkyl, COOH; more preferably R¹¹, R¹², R¹³, R¹⁴, R¹⁵ are independently selected from the group consisting of H, —(C₁-C₄) alkyl, —O(C₁-C₄)alkyl, —OH, Cl, —CO(C₁-C₄)alkyl, and COOH.

6. The quinone for use of claim 4 or 5, wherein the quinone is selected from quinone (1) wherein
(i) R¹³ is selected from the group consisting of —C(O)CH₃, -ethyl, -methyl, —OH, and —OCH₃, and R¹¹, R¹², R¹⁴, R¹⁵ are H or
(ii) R¹² is —C(O)CH₃, and R¹¹, R¹³, R¹⁴, R¹⁵ are H or
(iii) R¹¹ and R¹³ are methyl and R¹², R¹⁴, R¹⁵ are H.

7. The quinone for use of claims 1 to 3, wherein the quinone is selected from the group consisting of

[chemical structure]

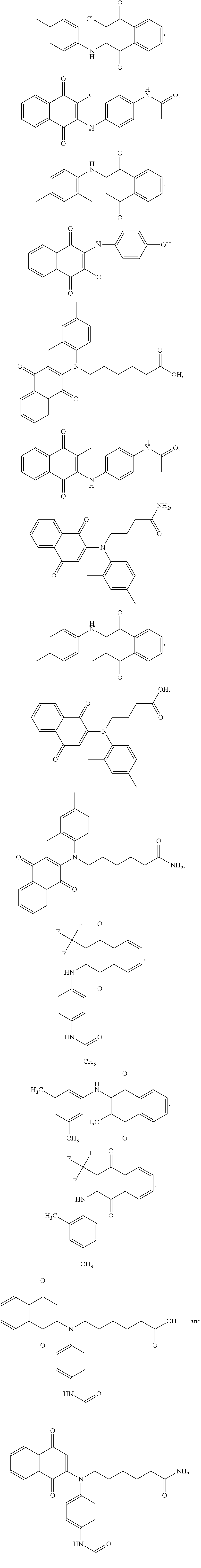

8. The quinone for use of claim 1 to 3, wherein the quinone is selected from the group consisting of

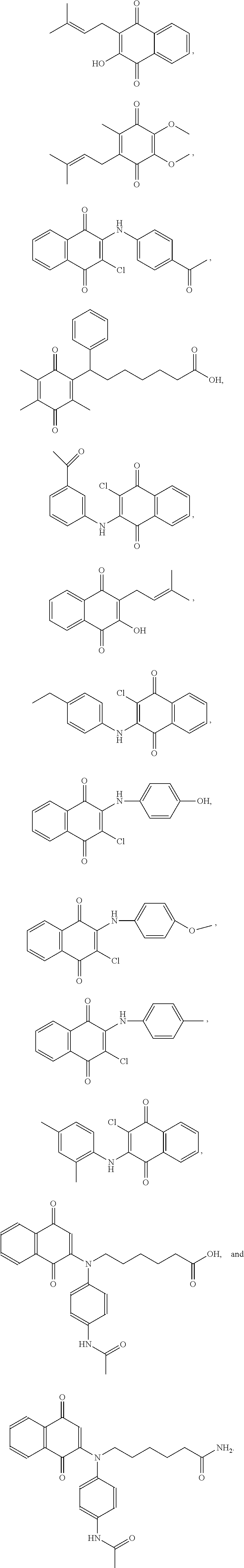

9. The quinone for use of claim 1 to 3, wherein the quinone is selected from the group consisting of

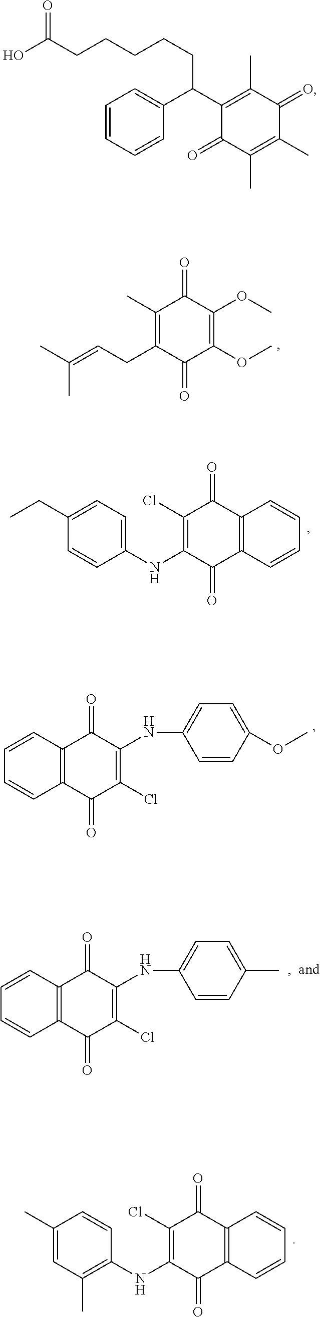

10. The quinone for use of claim 1 to 3, wherein the quinone is not

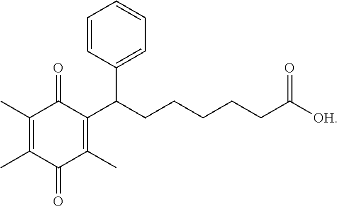 [SMILES: CC1=C(C)C(=O)C(C(CCCCCC(=O)O)c2ccccc2)=C(C)C1=O]

11. A quinone according to formula (III) 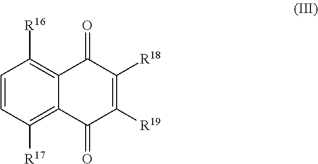
wherein
R¹⁶ and R¹⁷ are independently selected from the group consisting of H and —OH;
R¹⁸ is selected from the group consisting of H, —(C₁-C₃₀)alkyl, trifluoromethyl;
R¹⁹ is 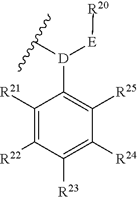
D is N;
E is a bond, (C₁-C₈)alkylene, or (C₂-C₈)alkenylene;
R²⁰ is selected from the group consisting of H, —COOH, —CONH₂, —COO(C₁-C₆)alkyl;
R²¹, R²², R²³, R²⁴, R²⁵ are independently selected from the group consisting of H, (C₁-C₄) alkyl, —O(C₁-C₄)alkyl, —OH, halogen, —CF₃, —CO(C₁-C₄)alkyl, COOH, —COO(C₁-C₆)alkyl, —CONH₂, —NH₂, —NHCO(C₁-C₄)alkyl, —NH(C₁-C₄)alkyl, and —N((C₁-C₄)alkyl)₂; preferably H, —(C₁-C₄) alkyl, —O(C₁-C₄)alkyl, —OH, Cl, F, —CF₃, —CO(C₁-C₄)alkyl, and COOH; more preferably H, —(C₁-C₄) alkyl, —O(C₁-C₄)alkyl, —OH, Cl, —CO(C₁-C₄)alkyl, and COOH;
wherein in (C₁-C₃₀)alkyl, one or more hydrogen atoms are optionally independently substituted by a substituent selected from the group consisting of —OH, —O(C₁-C₁₀)alkyl, (C₁-C₁₀)alkyl, —C(O)(C₁-C₁₀)alkyl, —COO(C₁-C₁₀)alkyl, —CONH₂, —COOH, —(C₆-C₁₂)aryl;
wherein the conditions i) and ii) are not present in the same molecule:
i) E is a bond, R²⁰ is H,
ii) R¹⁸ is H;
or a pharmaceutically acceptable salt, solvate, enantiomer, diastereomer, racemate or hydrate thereof.

12. The quinone of claim 11, wherein
R¹⁶ and R¹⁷ are independently selected from the group consisting of H;
R¹⁸ is selected from the group consisting of H, —CH₃, trifluoromethyl;
R¹⁹ is 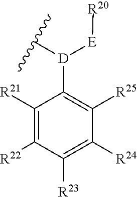
D is N;
E is a bond, (C₁-C₈)alkylene; preferably (C₃-C₅)alkylene;
R²⁰ is selected from the group consisting of H, —COOH, —CONH₂;
R²¹, R²², R²³, R²⁴, R²⁵ are independently selected from the group consisting of H, (C₁-C₄) alkyl, —NHCO(C₁-C₄)alkyl;
wherein the conditions i) and ii) are not present in the same molecule:
i) E is a bond, R²⁰ is H,
ii) R¹⁸ is H;
or a pharmaceutically acceptable salt, solvate, enantiomer, diastereomer, racemate or hydrate thereof.

13. The quinone of claim 11 or 12 selected from the group consisting of

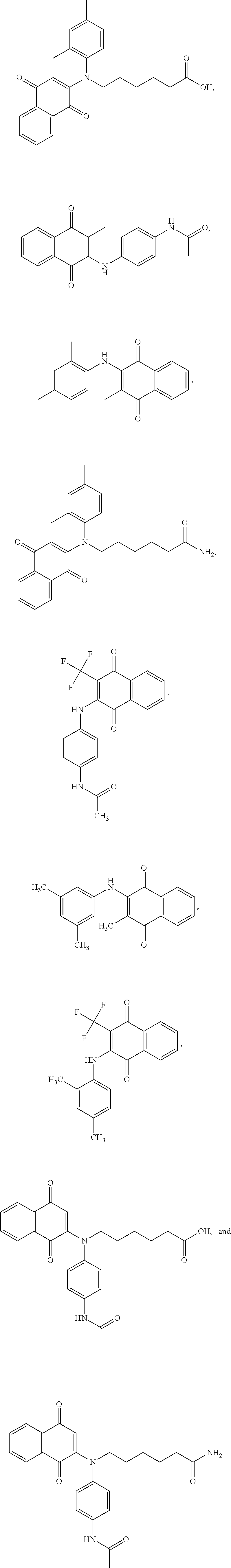

14. A pharmaceutical composition comprising the quinone for use as defined in claims 1 to 13 and a pharmaceutically acceptable carrier.
